(19) United States
(12) Patent Application Publication
(10) Pub. No.: US 2024/0261599 A1
(43) Pub. Date: Aug. 8, 2024

(54) LOW FREQUENCY MICRO AND NANOBUBBLES-ENHANCED ULTRASOUND MECHANOTHERAPY FOR NONINVASIVE CANCER SURGERY

(71) Applicant: RAMOT AT TEL AVIV UNIVERSITY LTD., Tel Aviv (IL)

(72) Inventors: Tali ILOVITSH, Tel Aviv (IL); Mike Gaston Natanael BISMUTH, Tel Aviv (IL); Bar GLICKSTEIN, Tel Aviv (IL)

(21) Appl. No.: 18/567,508

(22) PCT Filed: Jun. 8, 2022
(86) PCT No.: PCT/IL2022/050609

§ 371 Date: Dec. 6, 2023

Related U.S. Application Data
(60) Provisional application No. 63/208,981, filed on Jun. 10, 2021.

(51) Int. Cl. A61N 7/00

(52) U.S. Cl. CPC A61N 7/00; A61N 2007/0039; A61N 2007/0052; A61N 2007/0078

(57) ABSTRACT

Provided herein are systems, methods and compositions for noninvasive mechanical ultrasound (US) ablation of target tissue using microbubbles, nanobubbles or nanodroplets, combined with application of low frequency focused ultrasound.

### FIELD OF THE INVENTION

Provided herein are methods for noninvasive mechanical ultrasound ablation of tumors, using locally or systemically administered micro and/or nanobubbles followed by application of low frequency ultrasound. Further provided are methods for noninvasive mechanical ultrasound ablation of target tissue by administration of nanodroplets and conversion thereof, in-vivo, using imaging transducer(s) into microbubbles within target tissues.

### BACKGROUND

Focused ultrasound (US) is a versatile, noninvasive, clinically adopted therapy method to locally treat diseases via thermal or mechanical effects, by delivering powerful acoustic energy to a focal spot with a high spatiotemporal precision. These methods were implemented for the treatment of solid tumors deep within the body. Focused US (FUS) surgery offers less pain, shorter recovery time, and is noninvasive compared to surgical resection. In addition, it enables to treat patients that are otherwise ineligible for surgical resection. US surgery was used to treat various cancers, including pancreatic cancer, breast cancer, bone metastases, and liver and kidney tumors. FUS-mediated thermal ablation generates local temperature increase that can facilitate cell death at the target region. However, heat diffusivity and the need for precise thermal monitoring pose difficulties.

High-intensity focused ultrasound (US) is a non-invasive US technique in which an US beam is focused within the body to locally affect a targeted site. The two main methods of tissue ablation are thermal ablation and histotripsy. Thermal ablation uses heat to destroy a specific target tissue. The heated area is within the focal zone of the focused US beam, but due to heat diffusivity, the surrounding tissues risk being affected. Thus, thermal ablation requires precise monitoring, which typically involves using magnetic resonance thermometry. By contrast, histotripsy is a local, nonthermal US surgical method that uses short, high-intensity focused US energy to mechanically ablate deep tissues through cavitation, while leaving the surrounding healthy tissues unaffected. Histotripsy has been used for the treatment of conditions such as, liver cancer, thrombolysis, kidney stone erosion and benign prostatic hyperplasia. Although histotripsy is an approved technique, the main limitations arise from the high pressures that it requires (around 20 MPa). In terms of spatiotemporal precision, conventional histotripsy will fractionate any tissue within the focal zone. Therefore, it is highly susceptible to respiration motion that can lead to incomplete ablation or collateral damage. Another concern is the off-target effects. Another technological challenge is associated with the fabrication of high intensity focused transducers. Mechanical US surgery via histotripsy or mechanical ablations utilizes short and high intensity US pulses to mechanically destroy deep tissues, fractioning the targeted soft tissue into subcellular debris while leaving the surrounding organs and tissues unaffected. These mechanical effects result from the production of inertial cavitating bubbles or microscopic boiling bubbles. Inertial cavitation is a strong physical effect, where gas bubbles are formed, expand and violently collapse, excreting powerful mechanical effects on the surrounding tissue. Mechanical US surgery has been employed for the treatment of cancer by locally ablating tumors.

Seeded inertial cavitation using microbubbles (MBs) was proposed in an effort to reduce the pressure threshold required for mechanical US surgery. However, in the megahertz US range, the combination reduced the onset pressure to ˜10 MPa, which is still a high pressure.

NBs are considered sub-micron bubbles. Different NB shells and formulations exist, impacting their resulting diameter that typically varies between 100-700 nm. NB-mediated US was previously utilized for contrast imaging, gene delivery, molecular imaging, blood brain barrier opening for the delivery of small molecules, and synergistic thermal high intensity FUS ablation. Shen S, et. al. (Biomaterials. 2018; 181:293-306) discloses Folate-conjugated nanobubbles selectively target and kill cancer cells via ultrasound-triggered intracellular explosion, utilizing relatively large nanobubbles and relatively high intensity US. Owing to their small size, and since their main application is as contrast agents, NBs are typically coupled to high US frequencies (tens of MHz), on the order of their resonance frequency. This frequency is defined as the frequency at which the bubble first harmonic response has a local maximum. However, at this frequency, high-amplitude NBs oscillations are not observed, limiting the ability to obtain significant bioeffects as result of cavitation and the use of NBs for therapeutic applications that require strong cavitation.

The combination of nanoscale nanodroplets (NDs) and US has also been proposed to reduce the pressure required for standard histotripsy NDs are liquid bubbles that can vaporize into MBs when acoustically activated using US insonation. Standard NDs have a perfluorocarbon (PFC) liquid core stabilized by a shell that can be composed of lipids, polymers or proteins. In the context of mechanical US surgery, ND-mediated histotripsy was shown to be capable of forming a cavitation cloud while maintaining the effectiveness of regular histotripsy. Nevertheless, these methods still require high intensity ultrasound and high pressures on the order of ˜10 MPa (Vlaisavljevich et al., 2015, Ultrasound in Medicine and Biology Elsevier Inc., 2015; 41:2135-2147).

Thus, low energy and noninvasive mechanical US surgery with high spatial precision remains a grand challenge. Accordingly, there is an unmet need for improved therapeutic platforms that include systemic or localized administration of bubbles (microbubbles, nanobubbles and/or nanodroplets capable of being converted to microbubbles in situ), and application of low frequency US for efficient and safe ablation of tumors.

### SUMMARY

According to some embodiments, there are provided herein advantageous therapeutic platforms for the treatment of cancer, which utilize advantageous bubbles (MB) and/or nanobubbles (NB) that can be administered to a subject in need thereof, combined with application of low frequency ultrasound (US), to facilitate remote low energy US surgery of tumors, by triggering the MB or NB oscillations in target tissues, leading to mechanical effects on these tissues.

According to some embodiments, provided herein are improved therapeutic platforms and methods for low-energy, noninvasive, ultrasound (US)-based ablation of a target tissue that combines administration of bubbles (microbubbles (MBs) and/or nanobubbles (NBs)) to a subject, with Ultrasound US-based cavitation of the administered bubbles, using low-energy US, characterized by low frequency and low pressure. Advantageously, use of low-energy US provides a substantial reduction in mechanical disturbance of off-target tissue by the applied US compared to previously disclosed histotripsy methods, while simultaneously providing more potent ablation of target tissue.

In some embodiments, for convenience of presentation, the method in accordance with an embodiment of the disclosure may be referred to herein as “low energy bubble histotripsy” or “LE Bubble Histotripsy”.

According to some embodiments, advantageously, the MB may be administered locally, and the NBs may be administered systemically.

According to some embodiments, without wishing to be bound by any theory or mechanism, it is surprisingly demonstrated herein that exciting the bubbles disclosed herein, with US frequencies on the order of tens to hundreds of kilohertz, violent bubbles oscillations are triggered, thereby demonstrating the unique capability of the bubbles to serve as low-energy cavitation nuclei for histotripsy.

According to some embodiments, further provided herein are advantageous nanodroplets (NDs), composition comprising the same and uses thereof. NDs are microbubbles (MBs) that are compressed under low temperatures, thereby changing the state of matter of their gas core into liquid. Under ultrasound radiation, NDs can vaporize back into the gaseous phase. In some embodiments, the advantageous nanodroplets can be administered to the subject, and, and under application of high intensity ultrasound (in 1D, 2D/3D setting), can convert to microbubbles in/in close proximity to the target tissue, where after, low energy US can be applied to induce tissue damage. In some embodiments, the use of nanodroplets can induce volumetric damage to the target tissue, in particular, when utilizing a US system which includes a combination of 3D US imaging methods. According to some embodiments, the advantageous bubbles disclosed herein serve as an enhanced class of Ultrasound (US) theranostic contrast agents, while serving as low energy cavitation nuclei for US mechanotherapy of tumors. According to some embodiments, advantageously, coupling bubbles with low frequency US aids in reducing off target toxicity, while reducing the pressure threshold required for standard US surgery by an order of magnitude or more. This enables to overcome limitations that stem from the high intensity US levels that are generally used for histotripsy.

In further embodiments, the methods disclosed herein are endowed with the advantages of ultrasound (US), being safe, cost effective and clinically available, while the use of bubbles facilitate tumor targeting and alignment due to their ability to be visualized by US imaging. Furthermore, the use of low frequency US enhances penetration depth, minimizes distortion and attenuation and enlarges the focal spot compared to higher frequencies.

In some embodiments, advantageously, the methods and compositions disclosed herein, can successfully aid in treating deep-seated tumors and facilitate the treatment of larger tumor volumes simultaneously.

According to some embodiments, the method and compositions disclosed herein are suitable for treating various types of cancers and tumors, such as, but not limited to: solid tumors (including, for example, breast, lung, prostate, colon, pancreatic, liver, bone metastases, melanoma, bladder, kidney, sarcomas, carcinomas, Oral and oropharyngeal cancers, thyroid cancers, uterine cancers, neuroblastoma and lymphomas). In some embodiments, the cancer, is, for example, breast cancer tumors.

According to some embodiments, the bubbles and compositions disclosed herein, can further be utilized for noninvasive gene transfection via sonoporation (i.e., formation of small pores in cell membranes by using ultrasound).

According to some embodiments, there is provided herein a method for inducing damage to a target tissue of a subject, the method includes administering bubbles to the subject; and applying low frequency ultrasound (US) to the target tissue, to thereby induce damage to the target tissue.

According to some embodiments there is provided a method of treating cancer in the subject in need thereof, the method includes administering bubbles to the subject and applying low frequency US to the tumor region after a period of time.

In some embodiments, the bubbles are nanobubbles, and the administration is systemic. In some embodiments, where the bubbles are nanobubbles, the administration may be localized administration to or in close proximity to the target site/region.

According to some embodiments, the low-energy US (LE-US) may have a center frequency of about 1 MHz or less, 110 kHz or less, 105 kHz or less, 100 kHz or less, 90 kHz or less, 80 kHz or less, or about 80 kHz. In some embodiments, the LE-US may have a peak negative pressure (PNP) of about 550 kPa or less, 500 kPa or less, 450 kPa or less, 400 kPa or less, 350 kPa or less, 300 kPa or less, 280 kPa or less, 250 kPa or less, or about 250 kPa. In some embodiments, The LE-US may be characterized by having a mechanical index (MI) that is less than about 1.9, less than 1.85, less than about 1.8, less than 1.5, or less than 1, wherein the MI is calculated as equaling the PNP of the LE-US divided by the square root of the center frequency of the LE-US. In some exemplary embodiments, the LE-US may have a center frequency of 80 kHz or less and a PNP of 250 kPa or less.

Thus, according to some embodiments, there is provided an advantageous method for inducing damage to a target tissue of a subject, the method includes administering nanodroplets to the subject; applying high frequency ultrasound, to induce conversion of the nanodroplets to microbubbles; and applying low frequency ultrasound (US) to the target tissue, to thereby induce damage the target tissue.

In some embodiments, the high frequency US (HF-US) may have a center frequency of about 1 MHz or more, 2 MHz or more, 5 MHz or more, or about 5 MHz. The HF-US may be characterized by having a mechanical index (MI) that is less than 1.9, less than 1.5, or less than 1, wherein the MI is calculated as equaling the PNP of the HF-US multiplied by the square root of the center frequency of the HF-US. In some embodiments, the HF-US may be applied using a 1D ultrasound transducer. In some embodiments, the HF-US may be applied using a rotatory imaging probe and a therapeutic transducer, to thereby induce volumetric (3D) activation of the nanodroplets.

According to some embodiments, there is provided a method for inducing damage to a target tissue of a subject, the method includes: administering microbubbles (MB) and/or nanobubbles (NBs) to the subject; and applying low frequency ultrasound (US) having a peak negative pressure (PNP) of about 400 kPa or less to the target tissue, to thereby induce damage the target tissue.

According to some embodiments, the nanobubbles may have an average diameter in the range of about 50-250 nm. According to some embodiments, the nanobubbles may have an average diameter in the range of about 110-230 nm.

According to some embodiments, the nanobubbles may be administered systemically.

According to some embodiments, the microbubbles may have an average diameter in the range of about 250-3000 nm. In some embodiments, the MBs may have an average diameter in the range of about 700 nm to about 2000 nm.

According to some embodiments, the MBs may be administered locally, into or in the vicinity of the target tissue.

According to some embodiments, the microbubbles and/or nanobubbles may be essentially spherical.

According to some embodiments, the microbubbles and/or nanobubbles may include one or more lipids.

According to some embodiments, the MBs and/or NBs may include a targeting moiety on a shell thereof. In some embodiments, the targeting moiety may include a cell type-specific antibody conjugated to the shell.

According to some embodiments of the method, ultrasound is a low-energy US. In some embodiments, the US is in the frequency of less than about 1 MHz. In some embodiments, the frequency is less than about 200 KHz. In some embodiments, the US is in the frequency of less than about 100 KHz.

According to some embodiments, the US is characterized as having a peak negative pressure (PNP) of about 350 kPa or less.

According to some embodiments, the mechanical index of the US is about 1.9 or less.

According to some embodiments, the US may be applied after a time interval from the administration of the MBs and/or NBs. In some embodiments, the time interval is at least 10 minutes.

According to some embodiments, the tissue damage may include ablation, debulking and/or lesion of the tissue.

According to some embodiments, the target tissue may be or may include a tumor. In some embodiments, the tumor is a solid tumor. In some embodiments, the tumor is breast cancer.

According to some embodiments, there is provided a composition which includes microbubbles (MB) and/or nanobubbles (NBs) for use in inducing damage to a target tissue of a subject, wherein low frequency ultrasound (US) having a peak negative pressure (PNP) of about 400 kPa or less, is applied to the target tissue harboring said composition, to thereby induce damage to the target tissue.

According to some embodiments, the includes nanobubbles and is configured for systemic administration.

According to some embodiments, the composition may be formulated for localized administration.

According to some embodiments, there is provided a system for inducing damage to a target tissue of a subject, the system includes a low frequency focused ultrasound transmitter configured to emit low frequency ultrasound (US), having a peak negative pressure (PNP) of about 400kPa or less, towards a target tissue, wherein the subject has been administered with a composition comprising microbubbles (MB) and/or nanobubbles (NBs), and wherein said low frequency ultrasound causes the microbubbles and/or nanobubbles to induce damage to the target tissue.

According to some embodiments, there is provided a method for inducing damage to a target tissue of a subject, the method includes: administering nanodroplets to the subject; applying high frequency ultrasound (US) to the target tissue, to thereby form microbubbles in the target tissue; and applying low frequency US to the target tissue, to thereby induce tissue damage.

According to some embodiments, the high frequency US may be applied using an ultrasound imaging transducer comprising a plurality of transducing elements.

According to some embodiments, the high frequency US may be applied using a rotatory imaging US transducer, to thereby induce volumetric activation of the nanodroplets.

According to some embodiments, the high-frequency US is being characterized by a center frequency of 1 MHz or more and a mechanical index of less than about 1.9.

According to some embodiments, the imaging transducer may be situated within the therapeutic transducer.

According to some embodiments, the activation of the nanodroplets is facilitated in a 2-cycle excitation pulse. In some embodiments, the imaging transducer is configured to provide 2-cycle excitation pulse. According to some embodiments, the 2-cycle excitation pulse may have a center frequency of about 1 MHz or more, and a peak negative pressure (PNP) of over about 2 MPa. In some embodiments, the PNP is about 3.4 MPa.

According to some embodiments, the low frequency US is characterized as having a peak negative pressure (PNP) of about 400 kPa or less. According to some embodiments, the therapeutic transducer is configured to provide low frequency US having a PNP of about 400 kPa or less.

According to some embodiments, the nanodroplets may be administered systemically.

According to some embodiments, the low frequency US may be applied after a time interval from the administration of the NDs and/or after a time interval after application of the high frequency US.

According to some embodiments, the low frequency US may have a center frequency of about 1 MHz or less and the high frequency US may have a center frequency of about 1 MHz or more.

According to some embodiments, there is provided a system for inducing damage to a target tissue of a subject, the system includes a high frequency imaging transducer configured to provide high frequency ultrasound characterized by a center frequency of 1 MHz or more and a mechanical index of less than about 1.9, towards the target tissue, said target tissue comprises nanodroplets (NDs); and a low frequency focused ultrasound transmitter configured to emit low frequency ultrasound (US), having a peak negative pressure (PNP) of about 400 kPa or less, towards the target tissue; wherein said high frequency ultrasound facilitates conversion of nanodroplets in the target tissue to microbubbles, and wherein the low frequency ultrasound causes said microbubbles, to induce damage to the target tissue.

According to some embodiments, the imaging transducer may include an array of transducing elements.

According to some embodiments, the high frequency imaging transducer includes a rotatory imaging transducer configured to provide 3D ultrasound energy.

According to some embodiments, the imaging transducer may be located within the therapeutic transducer.

According to some embodiments, the system may further include one or more of: a user interface, a controller, a power supply, a communication unit, or any combination thereof.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1A—A schematic illustration of a method for afflicting local mechanical damage to target region, facilitated by microbubbles, according to some embodiments. Microbubbles are administered locally to a target region (Panel 1) (shown as a tumor); Low frequency focused ultrasound (“FUS”) is then applied to the tumor in order to implode the microbubbles (Panel 2), yielding local tumor mechanical damage (Panel 3);

FIG. 1B—A schematic illustration of a method for afflicting local mechanical damage to target region, facilitated by nanobubbles, according to some embodiments. Nanobubbles are systemically administered (Panel 1). Low frequency focused ultrasound (“FUS”) is then applied to the target region (shown as tumor) in order to implode the nanobubbles (Panel 2), to yield local tumor mechanical damage (Panel 3);

FIGS. 2A-2E show the results of theoretical predictions to compare MB expansion ratio under low center frequency insonation;

FIGS. 3A-3E show the results of a series of experiments evaluating MB cavitation dynamics as a function of various parameters of the US applied to the MBs;

FIGS. 4A-4C show the results of a series of experiments evaluating MB cavitation-induced cell death as a function of various parameters of the US applied to a mixture of MBs and a cancer cell line; and

FIGS. 5A-5F show results from an in vivo study testing embodiments of the LE bubble histotripsy method in accordance with an embodiment of the disclosure;

FIGS. 6A-6B. Nanobubble characterization: FIG. 6A—graph showing Nanobubbles (NBs) size distribution; FIG. 6B—Representative transmission election microscopy (TEM) images of NBs;

FIGS. 7A-7E. Characterization of low frequency nanobubbles insonation. FIG. 7A—Illustration of the setup used in the Characterization experiments: Agarose cube with a rod inclusion containing nanobubbles (NBs) or microbubbles (MBs) solution was placed at the focal region of a dual imaging-therapy setup; FIG. 7B—Ultrasound (US) images of the NB-filled inclusion pre and post US treatment with center frequencies of 250 kHz (1300 kPa) and 80 kHz (350 kPa); FIG. 7C—Normalized contrast reduction of NBs and MBs as a function of the applied peak negative pressures at a center frequency of 250 kHz; FIG. 7D—Normalized contrast reduction of NBs and MBs as a function of the applied peak negative pressures at a center frequency of 80 KHz; FIG. 7E–NB contrast reduction as a function of the mechanical index for 250 and 80 kHz insonation. All experiments were performed in triplicate. All data are plotted as mean±SD;

FIGS. 8A-8C—In vitro ultrasound-mediated nonthermal ablation. FIG. 8A—Illustration of the setup used in the experiments. Eppendorf tube containing a mixture of cancer cells and nanobubbles (NBs) was placed at the focal spot of the 80 kHz focused transducer. FIG. 8B—Impact of treatment duration on cell viability. FIG. 8C—Impact of NB concentration on cell viability. One-way ANOVA with Tukey's multiple comparisons test. Adjusted p values were **p<0.01, ***p<0.001, and ****p<0.0001. All experiments were performed in triplicate. All data are plotted as mean±SD;

FIGS. 9A-9B. NB tumor distribution using contrast harmonic ultrasound imaging in mice. FIG. 9A—Contrast pulse sequence ultrasound images of the tumor 0, 1, 4 and 10 minutes following a systemic injection of nanobubbles (NB). (Red) arrows indicate tumor borders. Images are presented with a 30 dB dynamic range. FIG. 9B—Tumor contrast enhancement as a function of time post NBs administration. All experiments were performed in triplicate. All data are plotted as mean±SD;

FIGS. 10A-10C—Nanobubble tumor extravasation. Contrast pulse sequence ultrasound (US) images of tumors following cardiac perfusion are shown: FIG. 10A—Sham tumor; FIG. 10B—Tumor that underwent cardiac perfusion 10 min following nanobubbles administration; FIG. 10C—Tumor that underwent cardiac perfusion 10 min after NB administration followed by additional 80 kHz US insonation to implode the tumor-accumulated NBs. Arrows indicate tumor borders. Images are presented with a 30 dB dynamic range;

FIGS. 11A-11C—Nanobubble-mediated low frequency insonation of breast cancer tumors in vivo. FIG. 11A—Nanobubbles (NBs) were systemically injected to tumor bearing mice. Next, tumors were insonated with low frequency ultrasound (US). Histological photomicrographs of tumor treated with low frequency US 10 min following systemic injection of: FIG. 11B—Microbubbles; FIG. 11C—NBs. Scale bars are 2 mm for tumor cross sections and 200 μm for 10× magnified images;

FIG. 12A—shows a schematic illustration of a two-step method for low energy nanodroplet (ND)-mediated histotripsy. The two-step method allows target tissue destruction using nanodroplets and conversion thereof to microbubbles within the target tissue, followed by application of low frequency ultrasound. As shown in FIG. 12A, nanodroplets (NDs) are administered to the target tissue (Panel 1). Thereafter, NDs are vaporized into gaseous microbubbles (MBs) using an imaging transducer (which can be a rotating imaging probe, when inducing 3D ND activation) applying high frequency focused ultrasound (FUS) (Panel 2). The generated MBs are then insonified at a low frequency US (Panel 3) to implode the MBs and cause mechanical/physical damage at the target tissue site (Panel 4);

FIG. 12B shows a pictogram of an exemplary testing system for concurrent 3D activation and detonation of NDs, according to some embodiments. The system includes a combination of rotatory imaging US transducer, configured to activate NDs, and a therapeutic transducer, configured to facilitate imploding of MBs formed by activation of the NDs, by transducing low frequency US;

FIG. 12C shows a schematic illustration of a US guided focused US system for the concurrent 3D activation and detonation of NDs, according to some embodiments. The system is used in a two-step method for volumetric tissue destruction using nanodroplets and conversion thereof into microbubbles within a target region, utilizing rotating imaging probe;

FIG. 13A shows a line graphs of a typical ND size distribution;

FIG. 13B shows a schematic illustration of a dual imaging-therapy setup used for optimization experiments of ND activation. In the optimization experiments, a ND solution is injected into a rod inclusion in an agarose phantom that is placed in the focal region of both the imaging and therapy transducers;

FIG. 13C shows a schematic illustration of a tissue mimicking phantom mold and the extracted phantom containing a well-shaped inclusion into which the diluted nanodroplet solution or the ex-vivo samples can be inserted;

FIG. 14 shows theoretical predictions for MB oscillations. The MB maximal expansion ratio as a function of the PNP for three low frequencies (850, 250, and 80 kHz) and for a MB's initial radius of 0.75 μm (750nm);

FIG. 15 shows lesion area quantification steps. In each image, the lesion area is outlined. The image is then converted into a binary image such that the interior of the lesion becomes black, while the rest of the image is white. The lesion area is the number of black pixels multiplied by the pixel area;

FIG. 16A-E—ND vaporization optimization results. Resulting inclusion contrast before and after ND vaporization as a function of: Activation duration (FIG. 16A); Activation pulse mechanical index (FIG. 16B); and ND concentration (FIG. 16C). FIG. 16D shows Exemplary US images before and after NDs activation for the different durations of 2, 5, and 10 seconds (tested under the conditions of FIG. 16A). FIG. 16E shows exemplary US images before and after NDs activation for different MI (1.25, 1.7, 1.8) (tested under conditions of FIG. 16B). All experiments were performed in triplicate. All data plotted as mean±SD;

FIGS. 17A-17B—show results of low frequency vaporized ND insonation optimization experiments: FIG. 17A—Contrast reduction as a function of PNP for vaporized NDs at the three center frequencies of 850, 250 and 80 kHz. FIG. 17B—Contrast reduction as a function of PNP for vaporized NDs compared to standard MBs at two center frequencies of 250 kHz and 80 kHz. The results were normalized by the maximum contrast reduction of each group. FIG. 17C—Examples of US images before and after application of a therapeutic US treatment with center frequencies of 850 kHz (either 390 or 870 kPa) 250 kHz (either 75 or 400 kPa) and 80 kHz (either 50 or 200 kPa). All experiments were performed in triplicate. All data are plotted as the mean±SD;

FIGS. 18A-18B show results from an ex-vivo study testing the effectiveness of a LE bubble histotripsy method in accordance with an embodiment of the disclosure, in which the MBs are derived from NDs;

FIGS. 19A-19B—Nanodroplet-mediated low energy histotripsy generates mechanical lesions in ex-vivo samples. FIG. 19A shows Histological photomicrographs of no treatment control (NTC), ND+only activation control, ND+only treatment control, ND−mediated histotripsy for ND vaporization followed by low frequency treatment at an MI=0.9 and center frequencies of 850, 250, and 80 kHz; FIG. 19B shows Quantification of the lesion area for each group. All experiments were performed in triplicate. Adjusted p values were **p<0.01, ***p<0.001, and ****p<0.0001. All data are plotted as the mean±SD;

FIG. 20 Shows Theoretical predictions for MB maximal expansion ratio as a function of the PNP at a center frequency of 105 kHz and for a MB's initial radius of 0.75 μm;

FIGS. 21A-21D show Optimization results for ND activation. Resulting inclusion contrast before and after ND vaporization as a function of: FIG. 21A—ND concentration; FIG. 21B—Activation duration; FIG. 21C—Activation pulse mechanical index. FIG. 21D shows Contrast reduction as a function of PNP for vaporized NDs at a center frequency of 105 kHz. All experiments were performed in triplicate. All data plotted as the mean±SD; and

FIGS. 22A-22B show Nanodroplet-mediated low energy histotripsy evaluation in ex-vivo samples. FIG. 22A—Histological photomicrographs and their binary images used for quantification of ND+only treatment control, ND+only activation control, ND−mediated histotripsy at a center frequency of 105 kHz and MI of 0.9 with 2D ND vaporization and 3D ND vaporization. FIG. 22B—Quantification of the lesion area for each group. All experiments were performed in triplicate. Adjusted p values were **p<0.01, ***p<0.001, and ****p<0.0001. All data are plotted as the mean±SD.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

According to some embodiments provided herein are advantageous bubbles, composition comprising the same and methods of using the same for US-surgery of tumors, by application of low frequency US. In further embodiments, provided herein are nanodroplets, compositions comprising the same and uses thereof for US-surgery of tumors, by combined application of high frequency Ultrasound to convert the nanodroplets to microbubbles, (in-situ), and application of low energy ultrasound to affect the microbubbles and include target tissue damage.

According to some embodiments, the term “bubbles” generally relates to microbubbles (MB) and/or to nanobubbles (NB). The term is directed to substantially spherical bodies having a shell (for example, a phospholipid shell) and a fluid core (for example, gaseous core), capable of serving as low energy cavitation nuclei for US mechanotherapy of target region (such as, tumor region). In some embodiments, the bubbles may be nanobubbles (i.e., have a diameter of up to about 350 nm) or microbubbles (i.e., have a diameter of between about 350 nm-3000 nm), as further detailed hereinbelow.

According to some embodiments, further provide herein are systems for US-surgery of tumors, which include one or more US transducers, capable of ultimately affect bubbles in target tissues, to include tissue damage. In some embodiments, the US transducers may be low frequency transducers and one or more additional high frequency transducers. In some embodiments, the high frequency transducers may include rotary imaging transducers that can be used in combination with application of nanodroplets, to ultimately induce increase, volumetric damage to the target tissue.

According to some embodiments, capabilities of the bubbles disclosed herein (such as, MBs and NBs) demonstrate their advantageous use for US-cancer surgery treatment of various cancers and tissues. For example, as exemplified herein, such capabilities of the MBs or NBs include uses thereof in US-cancer surgery in a breast cancer tumor model, as demonstrated in a mice model. According to some exemplary embodiments, following administration of the bubbles (for example, localized administration of microbubbles, or systemic NB administration (for example, by injection)), and coupled with low-frequency tumor insonation, the bubbles that are ultimately located in the tumor (or in close proximity thereto), are used as mechanical therapeutic warheads, creating large lesions in the tumor, at a significantly lower cavitation threshold compared to standard US surgery and advantageously with minimal off-target effects.

According to some embodiments, provided herein are noninvasive mechanical US ablation methods using a combination of administered bubbles and low frequency focused US. According to some embodiments, as exemplified herein, coupled with 80 kHz US insonation, NBs or MBs can serve as low energy cavitation nuclei for histotripsy, while reducing the energy required for standard US surgery by over an order of magnitude. The NB/MB-mediated US mechanotherapy can yield effective low energy US surgery of solid tumors.

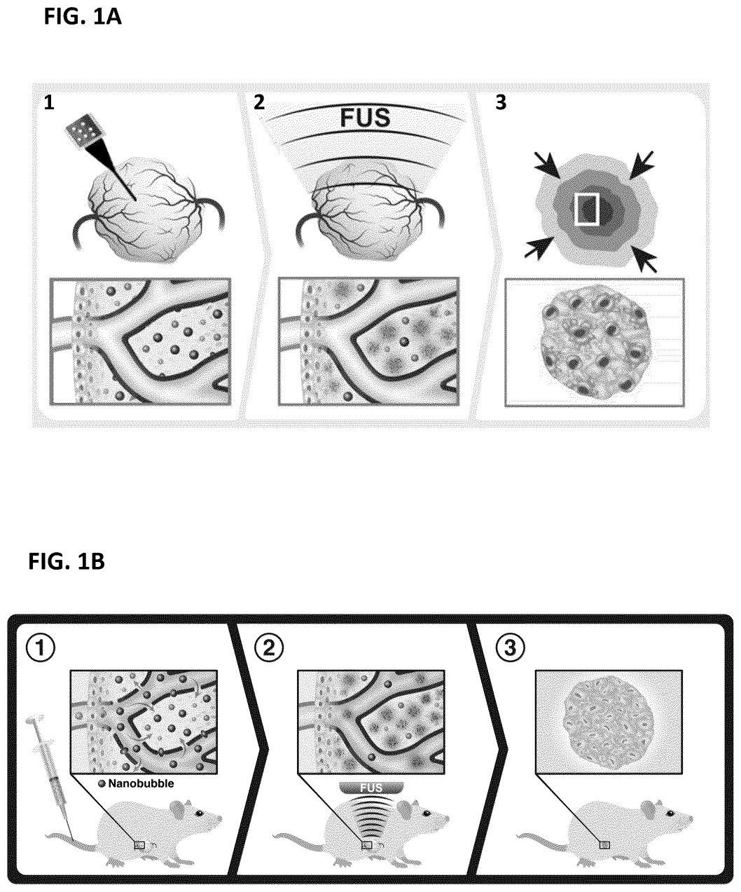

Reference is made to FIG. 1A, which is a schematic illustration of a method for afflicting local mechanical damage to target tissue, utilizing MBs, according to some embodiments. Microbubbles are locally administered (for example, by injection) to target tissue (tumor tissue) in a subject (Panel 1); Low frequency focused ultrasound (“FUS”) is then applied to the tumor in order to implode the microbubbles (Panel 2), to thereby yield local tumor mechanical damage (Panel 3).

Reference is made to FIG. 1B, which is a schematic illustration of a method for afflicting local mechanical damage to tumors, utilizing NBs, according to some embodiments. Nanobubbles are systemically administered (for example, by injection) to cancer bearing subject (exemplified as mice, panel 1); Low frequency focused ultrasound (“FUS”) is then applied to the tumor region in order to implode the nanobubbles (Panel 2); to thereby yield local tumor mechanical damage (Panel 3).

Thus, according to some embodiments, advantageously, the method disclosed herein utilizes a mechanical index (MI) within the safety limits. The MI parameter is defined as the peak negative pressure (PNP) divided by the square root of frequency, is a parameter that determines the likelihood of creating mechanical damage as a result of US application.

According to some embodiments, the MBs may have a diameter in the range of about 300-3000 nm. In some embodiments, the MBs may have an average diameter in the range of about 400-2500 nm. In some embodiments, the MBs may have an average diameter in the range of about 500-2000 nm. In some embodiments, the MBs may have an average diameter in the range of about 600-1800 nm. In some embodiments, the MBs may have an average diameter in the range of about 700-1500 nm. In some embodiments, the MBs may have an average diameter in the range of about 750-1200 nm. In some embodiments, the MBs may have an average diameter in of about 750 nm. In some embodiments, the MBs may have an average diameter of over 300 nm, over about 500 nm, over about 750 nm, over about 800 nm. Each possibility is a separate embodiment.

According to some embodiments, the NBs may have an average diameter in the range of about 25-300 nm. In some embodiments, the NBs may have an average diameter in the range of about 50-250 nm. In some embodiments, the NBs may have an average diameter in the range of about 75-200. In some embodiments, the NBs may have an average diameter in the range of about 90-150 nm. In some embodiments, the NBs may have an average diameter in the range of about 100-250 nm. In some embodiments, the NBs may have an average diameter of about 110-230 nm. In some embodiments, the NBs may have an average diameter in the range of about 150-200 nm. In some embodiments, the NBs may have an average diameter of about 170 nm. In some embodiments, the NBs may have an average diameter of less than about 300 nm, less than about 250 nm, less than about 200 nm, each possibility is a separate embodiment.

According to some embodiments, the low-energy US (LE-US) may have a center frequency of 1000 kHz or less, 800 kHz or less, 500 kHz or less, 200 kHz or less, 100 kHz or less, 90 kHz or less, 80 kHz or less, or about 80 kHz. In some embodiments, the US may have a peak negative pressure (PNP) of 550 kPa or less, 500 kPa or less, 450 kPa or less, 400 kPa or less, 350 kPa or less, 300 kPa or less, 280 kPa or less, 250 kPa or less, or about 250 kPa. In some embodiments, the US may be characterized by having a mechanical index (MI) that is less than about 1.9, less than about 1.5, less than about 1. In some embodiments, the MI may be calculated as equaling the PNP of the US divided by the square root of the center frequency of the US.

According to some exemplary embodiments, 80 kHz US may be applied, using an MI of 1.1-1.5 (for example, 1.3), burst length of about 0.5-10 ms (for example, about 1.56 ms), a PRF of about 10-100 Hz (for example, about 30 Hz), and a total treatment duration of about 1-10 minutes (for example, 2 minutes).

According to some embodiments, following a systemic injection and coupled with low frequency insonation, nanoscale nanobubbles with a mean diameter of about 170 nm, can serve as mechanical therapeutic warheads that trigger potent mechanical effects in tumors in a noninvasive and remote manner.

According to some embodiments, complete nanobubbles destruction may be achieved at a mechanical index of 2.6 for the 250 kHz insonation, as opposed to 1.2 for the 80 KHz frequency. Thus, the 80 kHz insonation comply with the safety regulations that require operation below a mechanical index of 1.9. In vitro, in breast cancer tumor cells, 80 kHz insonation of nanobubbles reduced cell viability to 17.3±1.7% of live cells, compared to control groups. In vivo, in a breast cancer tumor mice model, the disclosed method resulted in effective noninvasive mechanical tumor ablation and tumor tissue debulking, as observed via histology. This method provides a unique theranostic platform for safe, noninvasive and low energy tumor mechanotherapy.

According to some embodiments, the bubbles are lipid bubbles, having an external lipid shell. In some embodiments, the shell may include such components as, but not limited to: disteroylphosphatidylcholine (DSPC), 2-dibehenoyl-sn-glycero-3-phosphocholine (C22), 1,2-dipalmitoyl-sn-glycero-3-phosphate (DPPA), 1,2-dipalmitoyl-sn-glycero-3-phosphoethanolamine (DPPE), 1,2-distearoyl-sn-glycero-3-phosphoethanolamine-N-[methoxy(polyethylene glycol)-2000] (ammonium salt) (DSPE-PEG2K) and 1,2-distearoylsnglycero-3-phosphoethanolamine-N-[biotinyl(polyethylene glycol) 2000] (DSPE-PEG2000-Biotin). In some embodiments, the bubbles include a fluid core. In some embodiments, the fluid is gas. In some embodiments, the gas is selected from: perfluorobutane (C4F10), octafluoropropane C3F8, perfluorocarbons, sulfur hexafluoride, air and nitrogen.

In some embodiments, the microbubbles have an external lipid shell. In some embodiments, the lipid shell may include phospholipids. In some embodiments, the lipid shell of the microbubbles may include: (2.5 mg per 1 mL) disteroylphosphatidylcholine (DSPC), and 1,2-distearoyl-sn-glycero-3-phosphoethanolamine-N-[methoxy(polyethylene glycol)-2000] (ammonium salt) (DSPE-PEG2K). In some exemplary embodiments, for formation of targeted MBs (TMB) the lipids may further include 1,2-distearoylsnglycero-3-phosphoethanolamine-N-[biotinyl(polyethylene glycol) 2000] (DSPE-PEG2000-Biotin). In some embodiments, a molar ratio between the lipids may be 90:10, or 90:5:5.

In some embodiments, the surface tension of the MB outer radius may be in the range of about 0.03-0.5 N/m in saline. For example, the surface tension of the MB outer radius may be about 0.073 N/m (saline). In some embodiments, the surface tension of the MB inner radius may be in the range of 0.01-0.45 N/m. For example, the surface tension of the MB inner radius may be about 0.04 N/m. In some embodiments, the shell density of the MBs may be in the range of about 100-7000 Kg/m³. For example, the shell density of the MBs may be about 1000 kg/m³. In some embodiments, the shell shear modulus may be in the range of about 10-700 MPa. For example, the shell shear modulus may be about 122 MPa. In some embodiments, the shell viscosity may be in the range of about 0.5-5 Pa·s. For example, the shell viscosity may be about 2.5 Pa·s. In some embodiments, the shell surface dilatational viscosity may be in the range of about 1*10⁸ N−1*10¹⁰ N. For example, the shell surface dilatational viscosity may be about 7.2×10⁹ N. In some embodiments, the elastic compression modulus may be in the range of about 0.1-1 N/m. For example, the elastic compression modulus may be about 0.55 N/m. In some embodiments, the shell thickness may be in the range of about 0.5-2.5 nm. In some embodiments, the shell thickness may be about 1.5 nm.

In some embodiments, the nanobubbles have an external lipid shell. In some embodiments, the lipid shell may include phospholipids. In some embodiments, the lipid shell may include: 1,2-dibehenoyl-sn-glycero-3-phosphocholine (C22), 1,2-dipalmitoyl-sn-glycero-3-phosphate (DPPA), 1,2-dipalmitoyl-sn-glycero-3-phosphoethanolamine (DPPE), and 1,2-distearoyl-sn-glycero-3-phosphoethanolamine-N-[methoxy(polyethylene glycol)-2000] (ammonium salt) (DSPE-mPEG 2000). In some embodiments, the lipids may be in a molar ratio of 18.8:4.2:8.1:1 with a final lipid concentration of 10 mg/mL. In some embodiments, the lipid mixture may be sonicated.

According to some embodiments, the size distribution and concentration of freshly prepared MBs, and/or NBs may be measured using a particle sizing system. As exemplified herein, mean MB diameter may be about 500-900 nm (for example, 750 nm). As exemplified herein, mean NB diameter may be about 170 +60 nm. In some embodiments, the concentration of the bubbles may be 3.3×10¹² particles/ml. According to some embodiments morphology of MBs and/or NBs may be characterized using transmission election microscopy (TEM).

According to some embodiments, as exemplified herein, the bubbles (NBs/MBs) may exhibit a spherical morphology.

According to some embodiments, the bubbles (i.e., NBs or MBs) may further include a targeting moiety on an external region thereof. In some embodiments the targeting moiety may be on the shell of the bubbles. In some embodiments, the targeting moiety may be a cell-type specific targeting moiety. In some embodiments, the targeting moiety may be a cell-type specific antibody.

According to some embodiments, the amount/concentration/number of the microbubbles may be determined according to the target tissue, size of tissue, type of tumor, size of tumor, location of the tumor, and the like. In some embodiments, the amount of MBs administered may be about 1×10⁵ MBs. In some embodiments, the amount of MBs administered may be about 1×10⁶ MBs. In some embodiments, the amount of MBs administered may be about 5×10⁶ MBs. In some embodiments, the amount of MBs administered may be about 6.6×10⁶ MBs. In some embodiments, the amount of MBs administered may be at least about 1×10⁷ MBs. In some embodiments, the amount of MBs administered may be at least about 1×10⁸ MBs. In some embodiments, the amount of MBs administered may be at least about 1×10⁹ MBs. In some embodiments, the amount of MBs administered may be at least about 1×10¹⁰ MBs. In some embodiments, the concentration of MBs administered may be about 1×10¹¹ MBs/20 μl.

According to some embodiments, the amount/concentration/number of the nanobubbles may be determined according to the target tissue, size of tissue, type of tumor, size of tumor, location of the tumor, and the like. In some embodiments, the amount of NBs administered may be about 1×10¹¹ NBs. In some embodiments, the amount of NBs administered may be about 5×10¹¹ NBs. In some embodiments, the amount of NBs administered may be about 6.6×10¹¹ NBs. In some embodiments, the amount of NBs administered may be at least about 1×10⁶ NBs. In some embodiments, the amount of NBs administered may be at least about 1×10⁷ NBs. In some embodiments, the amount of NBs administered may be at least about 1×10⁸ NBs. In some embodiments, the amount of NBs administered may be at least about 1×10⁹ NBs. In some embodiments, the concentration of NBs administered may be about 1×10¹⁰ NBs/200 μl.

According to some embodiments, nanobubbles may be administered by systemic administration. In some embodiments, systemic administration may include, for example, parenteral administration, including, for example: intravenously, intra-arterially, intramuscularly, intraperitoneally, intradermally, intravitreally, or subcutaneously administration. In some embodiments, the systemic administration is by injection.

According to some embodiments, the application of US may be performed at a time period after administration of the bubbles (NBs or MBs). In some embodiments, the time period may be at least 5 minutes, at least 10 minutes, at least 20 minutes, at least 30 minutes, at least 60 minutes. Each possibility is a separate embodiment.

According to some embodiments, in order to characterize the acoustical behavior of NBs following low frequency insonation and confirm that low-energy US can result in NB inertial cavitation, contrast reduction experiments within tissue mimicking phantoms may be performed. Comparing MBs and NBs contrast reduction as a function of the applied PNP revealed that the pressure threshold required for maximal contrast reduction of NBs may be higher than that of MBs, for the two low frequencies tested. Importantly, the NB maximal contrast reduction was achieved at an MI of 2.6 for the 250 kHz, compared to 1.2 for the 80 kHz insonation. Therefore, while low energy MB-mediated mechanotherapy at an MI below 1.9 could be performed at either 250 or 80 kHz, for NBs, an 80 kHz insonation is used in order to maximize low energy cavitation effects, while complying with the FDA safety limits.

According to some embodiments, as exemplified herein, in vitro experiments in breast cancer cell cultures can be used to assess the bioeffects of NB-mediated low frequency insonation on cell viability. As shown in Example 3, cell viability was reduced to 17.3±1.7% of live cells for the highest NB concentration that was tested (12.5×10⁷ NBs/μL). No significant changes in cell viability were observed for the control groups that included NTC, only US and only NBs (with the same NB concentration of 12.5×10⁷ NBs/μL) It should be noted that a major difference exists between MBs and NBs in the context of in vitro assays, where close proximity to the cells is known to play an important role. Previously it was shown that since MBs tend to float, free MBs+80 kHz insonation did not affect cell viability. NBs do not float immediately, but rather move in a Brownian motion within the suspension. In vivo, free NBs that are accumulated within the tumor tissue are trapped and as a result can possess a similar behavior as targeted NBs.

According to some embodiments, both the NBs acoustical characterization experiments in tissue mimicking phantoms and the in vitro experiments may be used as perquisite steps to identify the optimal parameters that were later used in the in vivo experiments. As exemplified herein, in vivo experiments were carried in a breast tumor bearing mice model, with the goal of evaluating the mechanical bioeffects of NB-mediated low frequency insonation on the tumors. Initially, contrast harmonic US imaging was used to visualize and quantify the NB tumor distribution after a systemic NB injection. Maximal contrast increase was observed ˜1 min post injection and remained roughly constant throughout the 10 minutes post injection that were imaged. The contrast increase in the tumor following a systemic NB injection is a combination of the blood flow circulating NBs and the tumor extravasated NBs. In order to confirm the extravasation of NBs into the tumor tissue, cardiac perfusion was performed 10 min after NBs injection, followed by harmonic imaging of the collected tumors. This allows to eliminate the NB signal arising from the blood vessels and leave only the signal of the tumor-accumulated NBs. Contrast enhancement by 10.3±2.5 dB was detected compared to sham tumors. In order to confirm that the contrast increase stems from the NBs, 80 kHz US treatment was applied to the collected perfused tumors, to implode the NBs. A contrast reduction by 8.3±1.0 dB was detected following 80 KHz US application, confirming that the signal observed in the tumor arises from the presence of NBs in the tumor tissue following perfusion.

According to some embodiments, there is provided herein a method for inducing damage to a target tissue of a subject, the method includes administering microbubbles (MBs), or a composition including the same to the subject; and applying ultrasound (US) to the target tissue, to thereby damage the target tissue. In some embodiments, the administration is localized.

According to some embodiments, there is provided herein a method for inducing damage to a target tissue of a subject, the method includes administering nanobubbles (NBs), or a composition including the same, to the subject; and applying ultrasound (US) to the target tissue, to thereby damage the target tissue. In some embodiments, the administration is systemic.

In some embodiments, the tissue damage may include ablation, debulking and/or lesion of the tissue.

According to some embodiments, the target tissue is or comprises a tumor. In some embodiments, the tumor is a solid tumor.

According to some embodiments, the administration of the bubbles or nanodroplets may include various routes of administration. Exemplary suitable routes of administration include, but are not limited to intra-nasally, parenterally, intravenously, topically, localized, intra-tumor, or by inhalation. According to another embodiment, systemic administration of the composition is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention.

According to some embodiments, compositions formulated for injection may be in the form of solutions, suspensions, dispersions or emulsions in oily or aqueous vehicles, and may contain formulatory agents such as suspending, stabilizing, and/or dispersing agents.

According to some embodiments, the bubbles or nanodroplets compositions may be administered intravenously, and may thus be formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

In some embodiments, the administration is localized, for example, intratumorally (i.e., in the tumor).

According to some embodiments, provided herein are nanodroplets, which can be converted, in-situ to microbubbles. As used herein, Nanodroplets (NDs) are microbubbles (MBs) that are compressed under low temperatures, thereby changing the state of matter of their gas core into liquid. With US exposure under certain conditions, NDs can vaporize back into a gaseous phase to form MBs. As detailed and exemplified herein, the use of NDs provide various advantages. In some instances, MBs are too big to extravasate into the tumor. Therefore, MBs are most often administered intratumorally. By contrast, nanodroplets have a sufficiently small size for accumulation in tumors via the bloodstream: nanodroplets can extravasate across tumor's leaky vasculature and enter the interstitial space to directly target cancer cells. In certain embodiments, NDs may be systemically administered to a subject (for example, by injection into the bloodstream), allowed to accumulate at target tumors, then converted (“activated”) into MBs by HF-US exposure. After the NDs are converted to MBs, the MBs are exposed to LE-US to induce cavitation to perform LE bubble histotripsy.

According to some embodiments, NDs may be made from MBs. In some embodiments, MBs are prepared (for example, by combining lipids such as disteroylphosphatidylcholine (DSPC) and 1,2-distearoyl-sn-glycero-3-phosphoethanolamine-N-[meth-oxy(polyethyleneglycol)-2000] (ammonium salt) (DSPE-PEG2K) at a molar ratio of 90:10 mol/mol. A buffer mixture of glycerol, propylene glycol, and PBS (pH 7.4) with a volume ratio of (16:3:1) may be added to the lipids and sonicated at 62° C. The precursor solution may be saturated with perfluorobutane and agitated to from MBs. Next, a condensation procedure may be performed to phase change the MBs into NDs. In some embodiments, the NDs may have an average diameter in the range of about 50-500 nm. In some embodiments, the NDs may have an average diameter in the range of about 250-400 nm. In some embodiments, the NDs may have an average diameter of about 300 nm.

According to some embodiments, the nanodroplets may be used in the two-step histotripsy method for inducing mechanical effect on target tissue. The method includes the steps of providing the nanodroplets (for example, by systemic or other suitable route of administration) to a subject or a target tissue, providing high frequency ultrasound to convert the NDs to MBs, in situ (i.e., within the target tissue), and providing low frequency US to implode the MBs, to thereby induce damage to the target tissue.

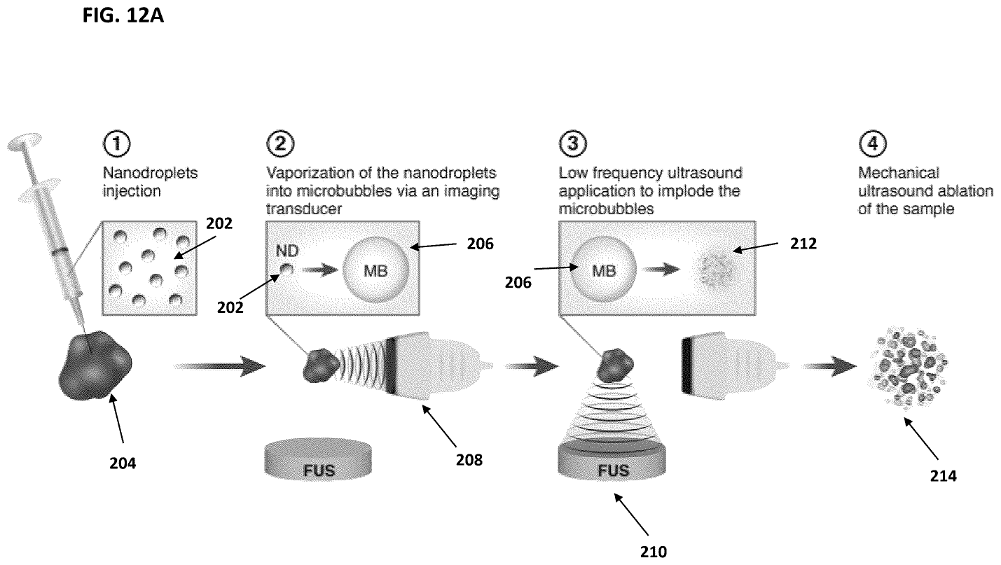

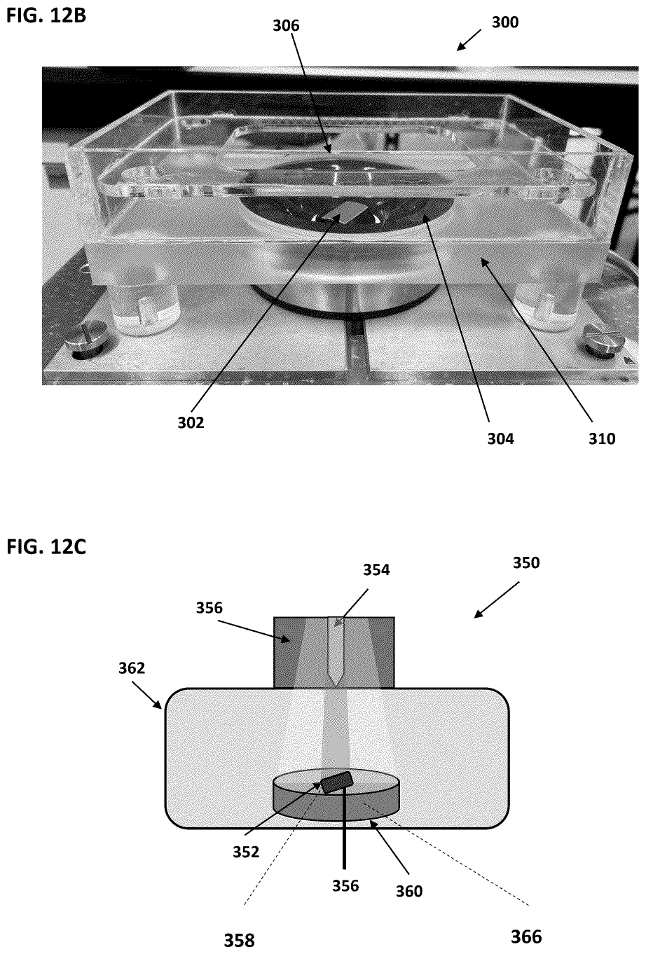

Reference is now made to FIG. 12A, which schematically illustrates a two-step method for LE bubble histotripsy, utilizing nanodroplets, according to some embodiments. As shown in FIG. 12A, in panel 1, nanodroplets (202) are administered to a target region (shown as tumor region 204), by localized administration or by systemic administration. The nanodroplets may be accumulated in the target area. As shown in Panel 2, the NDs are then allowed to vaporize into gaseous MBs 206, using an imaging transducer 208 (operating, for example, at a center frequency of 1-8 Mhz (for example, 5 MHz). As shown in Panel 3, the generated MBs 206, are then insonified at a low frequency (for example, below 250 kHz) by an ultrasonic transducer (FUS) 210, to implode the MBs (shown as imploded MBs 212) and facilitate significant mechanical/physical damage 214 at the target tissue site.

In some embodiments, as further detailed herein, both US steps may advantageously be performed, while operating below a mechanical index (MI) of 1.9. The MI is defined as the peak negative pressure (PNP) divided by the square root of the center frequency. Operation below a MI of 1.9 is beneficial in order to avoid undesired mechanical damage. Accordingly, the currently disclosed two-step method can be applied with minimal off target effects. The pressure required for ND vaporization at low frequencies is typically higher than at megahertz frequencies. Thus, the two-step method allows lowering the pressure threshold required for standard histotripsy by an order of magnitude or more.

Reference is now made to FIG. 12B, which shows a pictogram of a testing system for volumetric LE-bubble histotripsy, according to some embodiments. As shown in FIG. 12B, system 300 includes therapeutic transducer 302 and a rotatory imaging transducer 304, capable of providing 2D or 3D ultrasound to a target area 306, and being controlled by a motorized rotary. The transducers may be placed in a water bath 310. The imaging transducer may be controlled by a programmable US system. In some embodiments, the imaging transducer may include a plurality of transducing elements (i.e., an array of transducers). In some embodiments, by rotating the imaging transducer at a designated speed, acceleration, rotational position, velocity, timing, angle, etc., 3D activation of NDs may be facilitated. In some embodiments, the imaging transducer may transmit 2 cycle pulses on the location of the NDs (which are localized in the target area, as illustrated, for example, in FIG. 12A), to thereby activate the NDs. In addition, the imaging transducer may further be used for acquiring US images of the NDs before and after each testing. In some embodiments, the therapeutic transducer 302 may be any type of US transducer, such as, for example, a spherically focused single-element therapeutic transducer. Thus, after volumetric (i.e., in 3D) activation of the NDs by the imaging transducer and conversion thereof to MBs, the therapeutic transducer may be used to emit low frequency US to thereby cause the MBs to implode.

Reference is now made to FIG. 12C, which schematically illustrates a testing system for two-step method for volumetric LE bubble histotripsy, utilizing nanodroplets, according to some embodiments. As shown in FIG. 12C, system 350 includes at least one imaging transducer 352, capable of providing 3D ultrasound to a target area 354. The imaging transducer 352 may be moved/maneuvered manually, to provide ultrasound in 1D, 2D or 3D, or may be moved mechanically, via a dedicated platform, for example, by a motorized platform, exemplified as motorized rotary 356 in FIG. 12C. In some embodiments, the imaging transducer is a rotatory imaging array, having a plurality of transducer elements. In some embodiments, the rotatory imaging transducer may be controlled by a programmable US controller 358. The programmable US controller may include various modules, including, for example, PCc, power source, motor, and the like. The rotary transducer 352 may be situated within or in close proximity to a therapeutic transducer 360. The therapeutic transduced may be placed in a water tank 362. In some embodiments, the therapeutic transducer is operated using a suitable power output unit and controller (366). When used in experiments, the system may further include an agarose phantom 364, placed at the focal spot of both the imaging (352) and therapeutic (360) transducers. Depending on the experiment, the agarose phantom may contain either a diluted ND solution or an ex-vivo sample (such as, for example, chicken breast or liver sample), inside the target area 354 (also referred to as “rod inclusion”).

According to some exemplary embodiments, the testing system for two-step method for volumetric LE bubble histotripsy may include a rotating imaging array that is controlled by a motorized rotary and located at the bottom of a water tank. The imaging transducer (such as, for example, rotatory transducer IP104) may be controlled by a programmable US system (such as, Vantage 256, Verasonics Inc.) Such a transducer may include, for example, 128 elements, with an element size of 7 mm×0.283 mm (height×width), a kerf width of 0.025 mm and operates at a center frequency of 3.47 MHz. In some embodiments, 3D ND activation may be performed using such transducer by using the rotary motion while transmitting a 2 cycle pulse focused on the location of the administered (injected NDs) (distance of z=65 mm). The motorized rotary is an assembly that allows the user to rotate an imaging probe by +180° from its home position while examining a subject. The imaging probe rotary may be controlled via, for example, MATLAB, allowing a precise control over the rotational position, speed and acceleration of the attached imaging probe. The therapeutic transducer focus may be at a designated distance, such as, 60 mm. The transducer may be operated using a transducer power output unit (such as, TPO-200). In some embodiments, the PNP of both transducers (i.e., imaging transducer and therapeutic transducer) may be calibrated, for example, using a needle hydrophone (NH0200, Precision Acoustics, UK) in situ.

In some embodiments, the imaging transducer may also be used for acquiring US images of the NDs before and after activation, to analyze the properties of the NDs, and to allow optimization thereof.

According to some embodiments, for volumetric LE bubble histotripsy there is provided a hybrid platform (system) which includes an imaging transducer and a therapeutic transducer. In some embodiments, the imaging transducer may have a center frequency of 1 Mhz or more, and the therapeutic transducer may have a center frequency of 1 Mhz or less.

In some embodiments, the imaging transducer may include an array of transducers. In some embodiments, the imaging transducer may have a center frequency of at least about 1 Mhz, at least about MHz, at least about 2 MHZ, at least about 2.5 MHz, at least about 3 MHZ, at least about 3.5 MHz, at least about 3.7 MHz, at least about 4 MHZ, at least about 5 MHz. In some embodiments, the MI of the imaging transducer may be below about 1.9, below about 1.85, below about 1.7, below about 1.5, below about 1.2, below about 1. In some embodiments, the MI of the imaging transducer may be in the range of about 0.9-1.9. In some embodiments, the MI of the imaging transducer may be about 1.84. In some embodiments, the imaging transducer may transmit a 2-cycle pulse. In some embodiments, the imaging transducer may transmit a plurality of pluses. In some embodiments, the imaging transducer may be located/positioned with a therapeutic transducer. In some embodiments, the imaging transducer may operate for about 1-120 seconds, to exert an effect on the NDs. In some embodiments, the imaging transducer may operate for less than about 10 seconds. In some exemplary embodiments, the imaging transducer may operate for about 2 seconds.

In some embodiments, the therapeutic transducer may have a center frequency of about 850 kHz or less, about 500 kHz or less, about 250kHz or less, about 200 KHz or less, about 150 kHz or less, about 110 kHz or less, about 100 kHz or less, about 900 kHz or less, about 80 kHz or less, about 70 kHz or less. In some embodiments, the therapeutic transducer may have a center frequency of about 105 kHz. In some embodiments, the therapeutic transducer may have a PNP of about 1000 kPa or less, about 900 kPa or less, about 850 kPa or less, about 700 kPa or less, about 600 kPa or less, about 500 kPa or less, about 400 kPa or less, about 300 kPa or less, about 250 kPa or less. In some embodiments, the therapeutic transducer may have a PNP of about 290.

In some embodiments, the imaging transducer is a rotating (rotatory) imager, capable of providing US in 1D, 2D and/or 3D, depending on its position, moving capabilities, moving speed, acceleration, angle of movement, and the like. According to some embodiments, the operation of the rotatory imaging transducer may be controlled by a controller, configured to control any one of operating parameters of the transducer. According to some embodiments, when using a 1D transducer one line of activation is created. When using a rotating transducer, a plurality of lines of activation are created, such a plurality of lines of activation can generate 2D and/or 3D activation area, for example, by generating an essentially round circle of the activation area. According to some embodiments, the vaporized NDs are spread in a much wider area when using 2D/3D activation as compared to 1D activation.

According to some embodiments, use of 3D transducer can yield activation of a larger volume of vaporized NDs. According to some embodiments, using 2D US treatment resulted with an elongated lesion shape, and the 3D approach resulted with a round lesion shape. Thus, according to some embodiments, 3D activation allows the vaporized NDs to disperse over a larger area to thereby results in a greater destruction of the target tissue.

According to some embodiments, the concentration of administered ND may be in the range of about 1*10⁴ ND/ml−1*10^{p} ND/ml. In some exemplary embodiments, the ND concentration may be in the range of about 1*10⁷ ND/ml−5*10⁷/ml.

According to some embodiments, treatment duration (i.e., application of both US) may be in the range of 60-600 seconds. In some embodiments, duration of treatment may be about 90-80 seconds. In some embodiments, duration of treatment may be about 120 seconds.

In some embodiments, the high frequency US and low frequency US may be applied simultaneously. In In some embodiments, the high frequency US and low frequency US may be applied sequentially, with a time interval of 0.5-120 seconds therebetween.

Thus, according to some embodiments, there is provided a two-step method for low energy mechanical ultrasound surgery of tissues using nanodroplets, to reduce the required pressure threshold. In some embodiments, as detailed herein, a first step includes vaporizing the nanodroplets into gaseous microbubbles via megahertz ultrasound excitation. Then, low frequency ultrasound is applied to the microbubbles, which turns them into therapeutic warheads that trigger potent mechanical effects in the surrounding tissue. In some exemplary embodiments, as exemplified herein, optimal vaporization may be obtained when transmitting a 2-cycle excitation pulse at a center frequency of 5 MHz, and a peak negative pressure of 4.1 MPa (a mechanical index of 1.8). Low frequency insonation of the generated microbubbles at center frequency of 80 kHz, at a mechanical index of 0.9.

According to some embodiments, the two-step method disclosed herein may be used to inducing damage of a target tissue.

In some embodiments, the two-step method disclosed herein may be used for treating cancer in a subject in need thereof.

In some embodiments, there is provide a composition including NDs, for use in a two-step method for inducing target tissue damage.

In some embodiments, there is provided a system for facilitating two step method for inducing target tissue damage.

As used herein the term “about” refers to the designated value ±10%.

As used herein, the term “insonation” is directed to include treatment using ultrasound.

As used herein, the terms “low-energy US transducer” and “therapeutic transducer” may be used interchangeably. The terms relate to a US transducer configured to emit low energy US. In some embodiments, the low energy transducer is a Focused ultrasound.

In the description and claims of the application, the words “include” and “have”, and forms thereof, are not limited to members in a list with which the words may be associated.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1

### MB Modeling

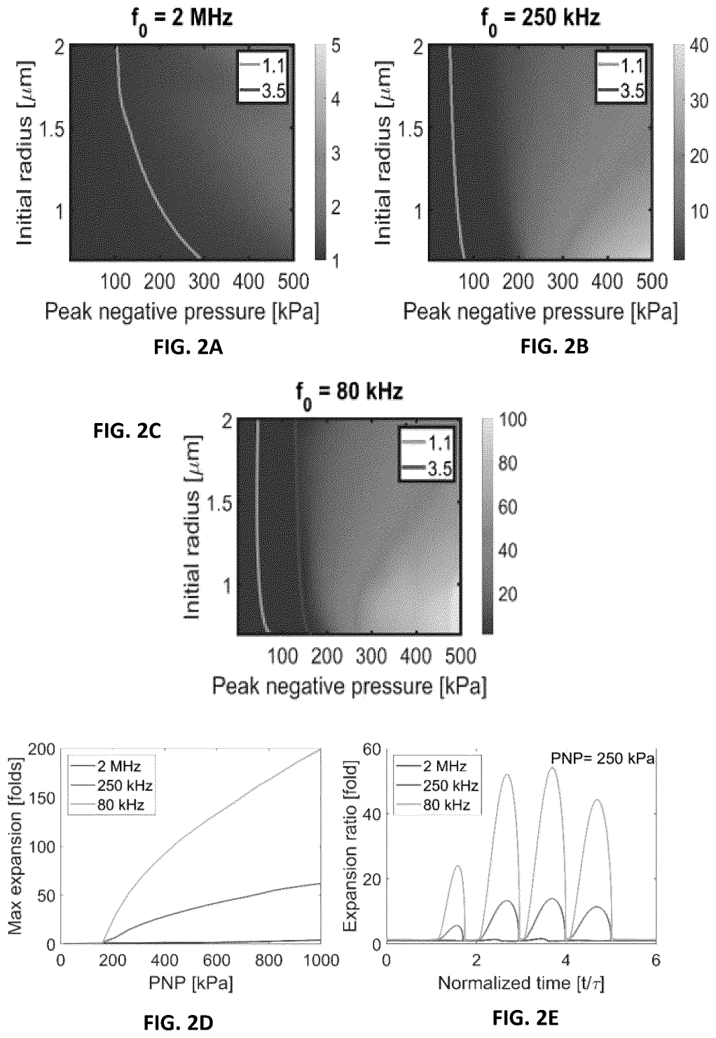

Reference is made to FIGS. 2A-2E, which shows the results of theoretical predictions using the Marmottant model to determine expansion ratios of oscillating MBs under US exposure (insonation), and comparing the calculated expansion ratio under low center frequency insonation (850 kHz, 250 and 80 kHz) and high frequency (2 MHz).

A Marmottant model as described in P. Marmottant et al. (“A model for large amplitude oscillations of coated bubbles accounting for buckling and rupture,” J. Acoust. Soc. Am., vol. 118, no. 6, pp. 3499-3505, 2005) was used to estimate MB oscillations and expansion ratio. Parameters such as MB composition, US excitation wave and the MBs' surrounding medium viscosity and density were taken into consideration in this model.

All simulations were performed in MATLAB (Mathworks, Natick, MA). The effect of center frequency, PNP and MB initial radius R₀ on oscillation behavior were evaluated. Initial MB radii values ranged between 0.75 to 2 μm. The expansion ratio for each MB initial radius was calculated as a function of varied PNP values between 0 to 1000 kPa. Simulations were performed for three center frequencies: 2 MHz, 250 kHz and 80 kHz. The surface tension of the MB outer radius was set to 0.073 N/m (saline) and to 0.04 N/m for surface tension of the inner radius. Shell density was 1000 kg/m³, shell shear modulus was 122 MPa, shell viscosity was 2.5 Pa·s, the shell surface dilatational viscosity was 7.2×10⁹ N and the elastic compression modulus was 0.55 N/m. Finally, shell thickness was set to 1.5 nm.

An expansion ratio is defined as the ratio between the maximum positive radius excursion and the minimum negative radius excursion of a ratio of an oscillating MB. The expansion ratio may be expressed as:

Max(D)/2R₀ (Formula 1)

Where Max(D) is the maximal MB diameter and R₀ is the resting radius.

MB oscillation and cavitation properties, including expansion ratio, caused by US application depend on certain US parameters. When exposed to US with low acoustic pressure, MBs tend to be compressed and expanded repeatedly in an oscillating process without disintegration or diminishment, in a process known in the art as “stable cavitation”. At higher acoustic pressures MB tend to undergo a process known in the art as “inertial cavitation”, in which the MBs disintegrate and fragment into smaller parts or diminish via gas diffusion. Inertial cavitation of a MB releases a substantially higher level of energy compared to stable cavitation, by way of example through induction of liquid jets than can cause acute mechanical damage to the surrounding tissue. In accordance with previous studies, stable cavitation was defined as MBs oscillating at an expansion ratio of between 1.1 and 3.5.

MB expansion ratio was predicted through numerical simulations at a range of parameters including peak negative pressure (PNP) ranging from 0 to 500 kPa, center frequencies of 2 MHZ (FIG. 2A), 250 kHz (FIG. 2B) and 80 kHz (FIG. 2C), and a range MB radii from 0.75 μm to 2 μm (to reflect the sizes of commercially available MBs such as SonoVue™ and Definity™). For each of FIGS. 2A-2C, the range of possible PNP values is represented in the X-axis and the range of initial MB radius values is represented in the Y-axis, and the degree of shading at any point in the x,y coordinate represents an expected expansion ratio of an MB having a given initial radius (x-axis) that is exposed to an US beam characterized by a given PNP (y-axis) and a given center frequency (2 MHz in FIG. 2A, 250 kHz in FIG. 2B, and 80 kHz in FIG. 2C).

For each US frequency, stable cavitation range that is associated with expansion ratios between 1.1 and 3.5 are indicated by solid gray and dashed white lines, respectively. It was found that lower US frequencies tended to produce higher expansion ratios. Out of the three US frequencies modeled in FIGS. 2A-2C, the highest expansion ratio values were predicted for the 80 kHz, reaching a factor of 100 at a PNP of 500 kPa. By comparison, the highest expansion ratio achieved by insonation with an US at 250 kHz and 500 kPa was 38, and the highest expansion ratio achieved by insonation with an US at 2 MHz and 500 kPa was 1.4. The stable cavitation range was narrowest (˜90 kPa) for 80 kHz US insonation, compared to ˜120 kPa for 250 kHz insonation and 460 kPa for 2 MHz insonation.

FIG. 2D shows the predicted expansion ratio of 0.75 μm radius MBs as a function of the PNP (0 to 1000 kPa) and the center frequency (2MHz, 250 kHz and 80 kHz) of the applied US. For a constant PNP of 250 kPa, FIG. 2E compared the expansion ratio as a function of time following 4-cycles excitation for the three different center frequencies. As shown in each of FIGS. 2C and 2D, lower US frequencies induced substantially higher expansion ratios in the MBs, even at frequency ranges below 250 kHz.

### Example 2

### In Vitro Parametric Testing of MB-to-US Interaction

For both in vitro (Examples 2, 3) and in vivo (Example 4) evaluations as described herein below, MBs and TMBs were produced as follows:

The MBs comprised a phospholipid shell and a perfluorobutane (C₄F₁₀) gas core. Lipids (2.5 mg per 1 mL) disteroylphosphatidylcholine (DSPC), and 1,2-distearoyl-sn-glycero-3-phosphoethanolamine-N-[methoxy(polyethylene glycol)-2000] (ammonium salt) (DSPE-PEG2K) (Sigma Aldrich) were combined at a molar ratio of 90:10 using a thin film hydration method to produce a phospholipid base. A buffer (mixture of glycerol (10%), propylene glycol (10%) and saline (80%) (pH 7.4)) were added to the phospholipid base and sonicated at 62° ° C. to produce a MB precursor solution. The MB precursor solution was aliquoted into vials with liquid volume of 1 mL and saturated with perfluorobutane. At the time of use, the vials were shaken for 45 sec in a vial shaker to induce creation of MBs within the MB precursor solution and the solution was purified via centrifugation to remove MBs smaller than 0.5 μm in radii.

The TMBs were prepared as follows: Lipids (2.5 mg per 1 mL) disteroylphosphatidylcholine (DSPC), 1,2-distearoyl-sn-glycero-3-phosphoethanolamine-N-[methoxy(polyethylene glycol)-2000] (ammonium salt) (DSPE-PEG2K) (Sigma Aldrich), and 1,2-distearoylsnglycero-3-phosphoethanolamine-N-[biotinyl(polyethylene glycol) 2000] (DSPE-PEG2000-Biotin), were combined at a molar ratio of 90:5:5 and prepared similarly to the untargeted MBs to produce a MB cake. Following activation via Vialmix and purification, 400 μg of streptavidin was added to the MB cake and incubated for 25 minutes at room temperature on a rotator. Next, the streptavidin-modified MBs were purified to remove excess streptavidin. Subsequently, 15 μg of biotynilated anti-mouse CD326 (EpCAM, BioLegend #118203) antibody was added to the streptavidin-MB cake followed by incubation on a rotator and purification as described in the precedent step.

The size and concentration of the purified MBs and TMBs were measured with a particle counter system (AccuSizer® FX-Nano, Particle Sizing Systems, Entegris, MA, USA). The MBs and TMBs were used within three hours of preparation. The size distribution and concentration of the MBs and TMBs changed by less than 5% between measurements.

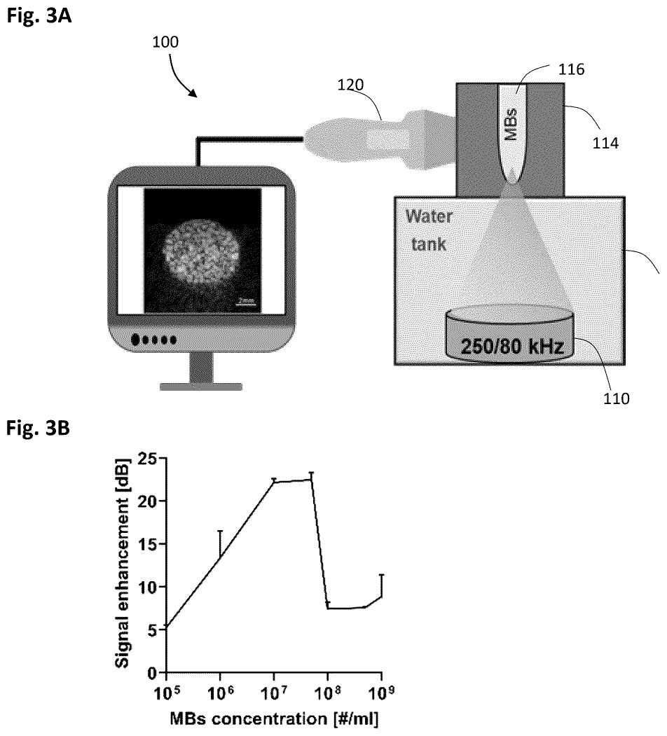

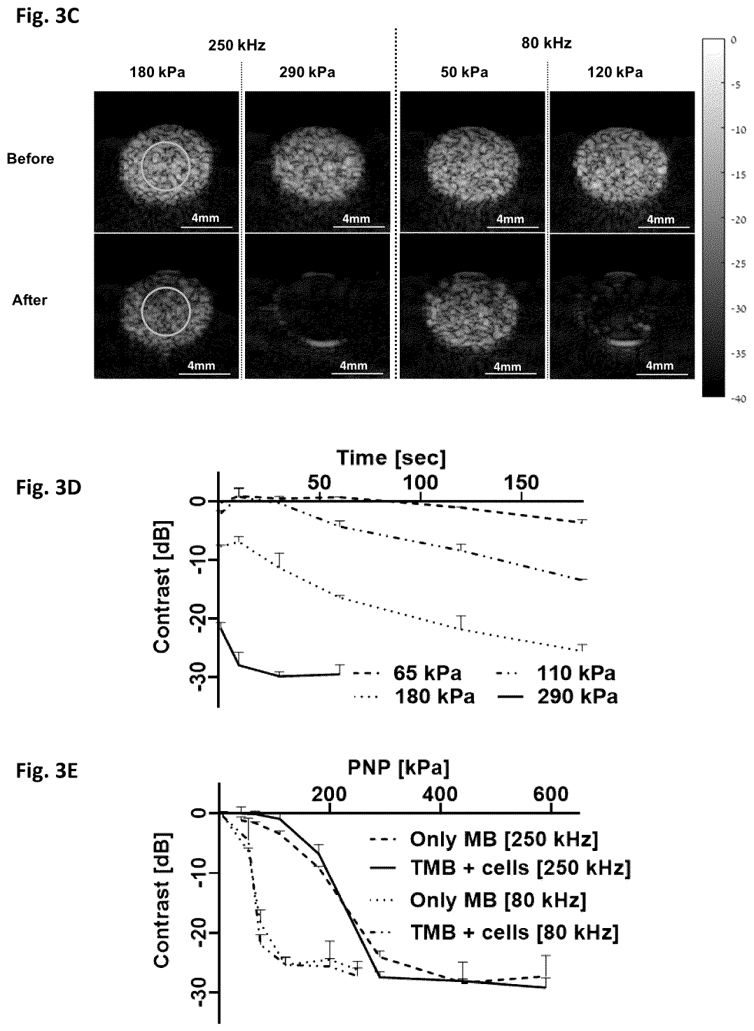

FIG. 3A schematically shows an experimental US insonation setup 100 comprising a 64 mm diameter spherically-focused single-element US transducer 110 (H117, Sonic Concepts, Bothell, WA, USA) operating either at 250 or 80 kHz center frequency using a designated matching network. The US transducer was placed at the bottom of a degassed water tank 112 facing upwards and focused to a distance of 45 mm, and an insonation target was placed at the focal spot of the focused US generated by the US transducer. The insonation target was either an agarose control block 114 (“phantom”) containing MBs or a 0.5 mL Eppendorf tube with breast cancer cells (not shown). The US transducer pressure was calibrated with a wideband needle hydrophone (NH0500, Precision Acoustics, UK; not shown). A transducer power output unit (not shown) combining a waveform generator (not shown) together with a radio frequency (RF) amplifier (TPO-200, Sonic Concepts, Bothell, WA, USA; not shown) was used to generate the desired RF signal consisting of 125-cycles of a sinusoid with a 250 kHz or 80 kHz center frequency and a pulse repetition time of 30 ms between the pulses. An imaging transducer 120 (such as, for example, L7-4, Philips ATL) controlled by a programmable US system (Verasonics, Vantage 256, Verasonics Inc., Redmond, WA, USA; not shown) was used to image the MB phantom before and after the application of the low frequency therapeutic US. Imaging transducer 120 was placed perpendicularly to spherically focused US transducer 110.

Parametric experiments with the agarose phantom with MBs or TMBs using the setup shown in FIG. 3A, and whose results are shown in FIGS. 3B-3E, were performed as follows:

Agarose powder (Sigma Aldrich) was mixed with deionized water to a 1.5% solution at ambient temperature and heated until all powder was completely dissolved. The solution was then poured into a mold and cooled at ambient temperature. The mold was 3D printed and contained a 6 mm rod inclusion 116. The phantom was placed at the focal spot of US insonation setup 110. In each experiment, a mixture of MBs or TMBs bound to cells were diluted in degassed phosphate buffered saline (PBS) and injected into rod inclusion 116. Imaging transducer 120 was used to image the MB phantom before and after the application of the low frequency US to determine contrast present in an image of the MBs captured by imaging transducer 120. The contrast of the image is defined as the difference in brightness before and after US treatment at the region of interest and is defined according to the formula:

$$Contrast [ dB ] = 20 {log}_{10} ( \frac{{μ}_{i}}{{μ}_{o}} ) \qquad ( Formula 2 )$$

where μᵢ is the mean pixel value of a region-of-interest (ROI) in an image of the MBs captured after US insonation, and μₒ is the mean pixel value of the same region before US treatment.

When MBs undergo inertial cavitation, they are destroyed and the degree of contrast in an image of the MBs is reduced as a result. To evaluate contrast reduction as a function of the PNP and center frequency of the applied US, the dual imaging-US setup as illustrated in FIG. 3A was used. Initially, MB concentration was optimized for maximum contrast. As shown in FIG. 3B, contrast increased with MB concentration up to a maximum contrast of about 23 dB between 1×10⁷ MBs/ml and 5×10⁷ MBs/ml, after which further increases in MB concentration led to a sharp fall in image contrast. Based on the above-noted result a concentration of 1×10⁷ MBs/ml was used for subsequent experiments.

Reference is made to FIG. 3C. To assess the impact of the high and low frequency US excitation on MB contrast reduction, an image of the MBs were captured before and after a 1 second US treatment. As shown in the figures, application of US having a higher PNP and/or low frequency tended to result in more pronounced contrast reduction due to induction of inertial cavitation. For example, contrast reduction of over 20 dB is observed after exposure to US having a PNP of 290 kPa at 250 kHz (high PNP/high frequency), as well as after exposure to US having a PNP of 120 kPa at a frequency of 80 kHz (medium PNP/low frequency). By contrast, treatment with US having a PNP of 180 kPa at 250 kHz (medium PNP/high frequency) and with US having a PNP of 50 kPa at 80 kHz (low PNP/low frequency) resulted in a substantially less pronounced contrast reduction of 9.1 dB and 5.8 dB, respectively.

The effect of US exposure on MBs and biological materials can be quantified in a number of ways that describe or predict the physical effects of the US exposure. A Mechanical index (MI), is a measure of a mechanical effect of an applied US in biological materials, and is defined as the peak negative pressure (PNP) of the US divided by the square root of the center frequency (CF) of the US according to the following formula (equation):

MI=PNP*(CF\hat{ }0.5) (Formula 3)

MI is a parameter used for clinical safety assessment of US. MI indicates the likelihood of adverse mechanical bio-effects (streaming, cavitation, etc.), by gauging the PNP for a given US frequency. MI can also be used to define safety thresholds for therapeutic uses of US by regulatory bodies. By way of example, the United States Food and Drug Administration (FDA) requires that US applied to a subject for the purposes of diagnostic imaging must have a MI below 1.9. Beyond this value, potential tissue damage is expected due to cavitation even without the presence of MBs.

Cavitation index (CI) provides a measure of potency of a US stimulation to induce inertial cavitation of MBs, and is defined as the PNP divided by the center frequency according to the following formula:

CI=PNP/CF (Formula 4)

CI serves as an indicator of likelihood whether the MB engages in stable cavitation or inertial cavitation. A CI above 0.02 indicates increased likelihood that the MB oscillation results in inertial cavitation.

Reference is made to FIG. 3D, which shows a line graph showing the induction of inertial cavitation as indicated by a reduction of image contrast (y-axis), as a function of time of exposure (x-axis) to US. In the study, US to which the MBs were exposed had a center frequency of 250 kHz. Each line represents different PNPs: 65 kPa is shown as a dashed line, 110 kPa is shown as a dash-dot-dot line, 180 kPa is shown as a dotted line, and 290 kPa is shown as a solid line. For lower PNPs (65 kPa, 110 kPa and 180 kPa), the graph reveals that contrasts decreases in a largely linear manner as a function of the treatment duration. For example, treatment with 65 kPa US for 180 seconds resulted in a contrast reduction of 3.6 dB, and treatment with 110 kPa US for 180 seconds resulted in a contrast reduction of 13.4 dB. By comparison, treatment with a high PNP of 290 kPa US resulted in non-linear contrast reduction, with the contrast being reduced by 21.6 dB within the first second of US exposure then leveling off at about −30 dB.

Reference is made to FIG. 3E, which shows the degree of inertial cavitation induction as indicated by a reduction of image contrast (y-axis) as a function of PNP (x-axis) of the applied US. MBs or a mixture of TMBs bound with 4T1 cells were exposed to 1 second of US at a range of PNPs up to 600 kPa, at a frequency of either 250 kHz or 80 kHz. It was shown with both MBs and TMB-bound 4T1 cells that a 1 second pulse of US at 80 kHz was substantially more potent in inducing inertial cavitation. Compared to a 1 second US treatment of 250 kHz US, a 1 second treatment with 80 kHz US resulted in contrast being reduced to a minimal value of −25 dB at a substantially lower PNP.

### Example 3

### In Vitro US-Mediated Ablation Assay for Carcinoma Cell Line

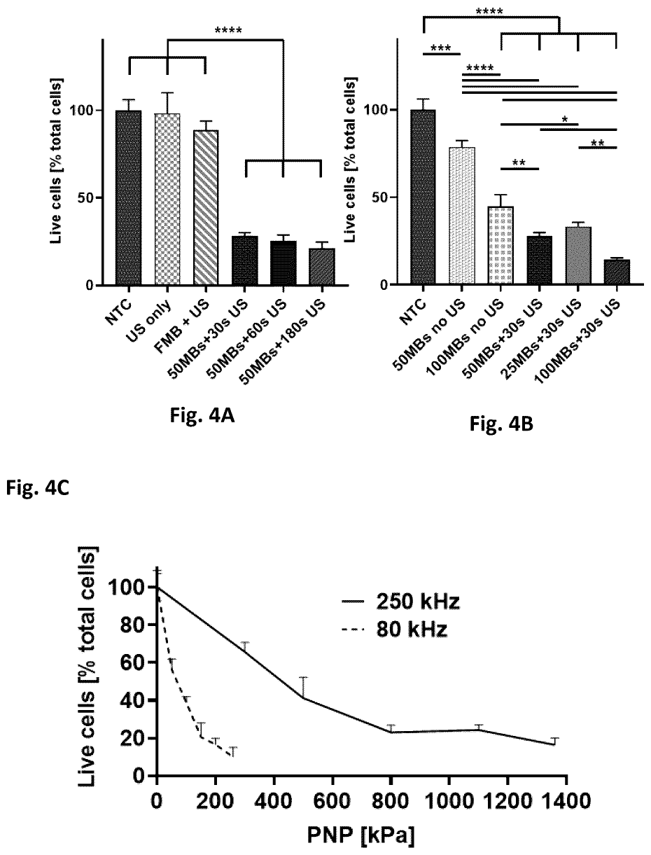

Reference is made to FIGS. 4A-4C, which show the results of in vitro ablation assays of a cancerous cell line treated with TMBs and low frequency US. Low frequency insonation-mediated in vitro experiments assessed the impact of TMB oscillations/cavitations on cell viability as a function of PNP and center frequency, and was used to determine MB concentration and US parameters capable of inducing the death of cancerous cells. In vitro US-mediated ablation assays using the setup shown in FIG. 3A and whose results are shown in FIGS. 4A-4B were performed as follows:

4T1 cells, a highly metastatic triple negative murine breast carcinoma cell line (purchased from ATCC) was used for the in vitro analysis. The 4T1 cells were cultured in RPMI 1640 supplemented with 10% v/v fetal bovine serum, 1% v/v penicillin-streptomycin and 0.292 g/L L-glutamine and grown in T75 tissue culture-treated flasks until about 85% confluency on the day of the experiment. The 4Tl cells were then collected via dissociation with TrypLE™ Express (Gibco Corp., 12604-013, Grand Island, NY, USA) and resuspended at 1×10⁶ cells in 300 μL degassed PBS containing calcium and magnesium (PBS+/+). The TMBs were added to the cell mixture at one of three concentrations (25 TMBs/cell, 50 TMBs/cell, or 100 TMBs/cell) and incubated for 20 minutes at room temperature on a rotator allowing the TMBs to bind to the cells. Following incubation, the mixture of cells and TMBs was aliquoted into 0.5 mL Eppendorf tubes. Finally, degassed PBS+/+ was added to a final volume of 0.48 mL per tube and incubated at room temperature for 30 minutes prior to the US treatment. Next, each Eppendorf tube was placed at the focal spot of the US setup and treated according to the different US treatment parameters tested. Sonication in all the in vitro studies consisted of a 125-cycles sinusoid with a 250 kHz or 80 kHz center frequency and a pulse repetition time of 30 ms. After treatment, cells were transferred to a six-well tissue culture dishes already containing RPMI 1640 complete medium supplemented with 2.5% v/v penicillin-streptomycin. Cells were cultured at 37° C. in a humidified 5% CO₂ incubator for 72 hours and were collected in 500 μL of TrypLE™ Express. Hemocytometry with Trypan Blue dead cell exclusion was used to assess viable cell number. All treatments were analyzed in triplicate.

As shown in FIG. 4A, the setup shown in FIG. 3A was used to determine an advantageous US exposure duration for inducing the death of 4T1 cells. T41 cells were exposed to US having a center frequency of 250 kHz and PNP of 500 kPa under various conditions. It was found that US exposure only (“US only”; checkered bar), as well as combining US exposure with application of free MBs (“FMB+US”; diagonal stripe bars) were ineffective in inducing cell death and not significantly different from untreated control (“NTC”; dark checkered bar). By contrast, combined treatment of US exposure (at 250 kHz and 500 kPa) together with TMB application (at a ratio of 50 TMB/cell) potently induced death of T41 cell. A 30 second exposure to US was as potent as 60 second and 180 second exposures: each of the three US treatment times reduced cell viability to 24.8% of live cells (p<0.0001. FIG. 4A).

As shown in FIG. 4B, the setup shown in FIG. 3A was used to determine an advantageous MB concentration inducing the death of 4T1 cells. In all conditions shown in FIG. 4B where US exposure was included, the US had a center frequency of 250 kHz and a PNP of 500 kPa, and treatment duration of 30 seconds was used. The constant US parameter were combined with different TMB concentrations (25, 50 or 100 TMBs per cell) to assess the effect of TMB concentration on 4T1 cell death. When combined with US exposure, increasing of TMBs concentration per cell led to more pronounced 4T1 cell death: a concentration of 100 TMBs per cell resulted in only 14±0.8% of 4T1 cell surviving the treatment, as compared to 33.4±2.3% viability (p<0.01) after being treated with 25 TMBs per cell. However, it was found that TMB treatment, at sufficient concentrations even without US stimulation, resulted in 4T1 cell death. By way of example, a control group (“100 MBs no US”) of 4T1 cells that was treated with TMBs at a concentration of 100 TMB per cell without US exposure, resulted in 4T1 viability being reduced to 44.9±6.5% after treatment (p<0.0001 compared to the non-treated control group “NTC”). In comparison, a control 50 TMB/cell without US resulted in post-treatment cell viability of 78±4% (p<0.0001 compared to the treated group), and 50 TMB/cell combined with the US treatment led to a post treatment cell viability of 28.2±1.8% viability. Therefore, a concentration of 50 TMBs per cell was selected for subsequent experiments.

As shown in FIG. 4C, cell viability after combined TMB and US treatment was compared between two US frequencies: 250 and 80 kHz and a range of PNPs. Under both treatment conditions, the other treatment parameters were identical: 4T1 cells were treated with TMBs at a concentration of 50 TMBs per cell, and was exposed to US having a PNP of up to 1400 kPa for a duration of 30 seconds. The graph shown in FIG. 4C plots the percentage of viable cells remaining after treatment (y-axis) against the PNP of the US treatment (x-axis), for each of the two tested frequencies: 80 kHz (dashed line) and 250 kHz (solid line). Five PNPs were tested for each US frequency, ranging from 300 kPa to 1360 kPa for 250 kHz and ranging from 50 kPa to 260 kPa for 80 kHz. As shown in the graph, 80 kHz US exposure caused substantially more robust reduction in cell viability compared to 250 kHz at all PNP ranges. By way of example as shown in FIG. 4C, at 250 kHz, a PNP of 800 kPa was needed to achieve post-treatment cell viability of 22.9±3.8% while only 150 kPa was required to achieve a similar degree of cell death using US of 80 kHz. Also shown in FIG. 4C, at a center frequency of 80 kHz, even a PNP of 50 kPa was sufficient to induce substantial cell death, with a post-treatment cell viability of about 60%. By contrast, at a center frequency of 250 kHz, 50 kPa would not have measurable resulted in any induced cell death.

### Example 4

### In Vivo US and MB-Mediated Breast Cancer Ablation Assay

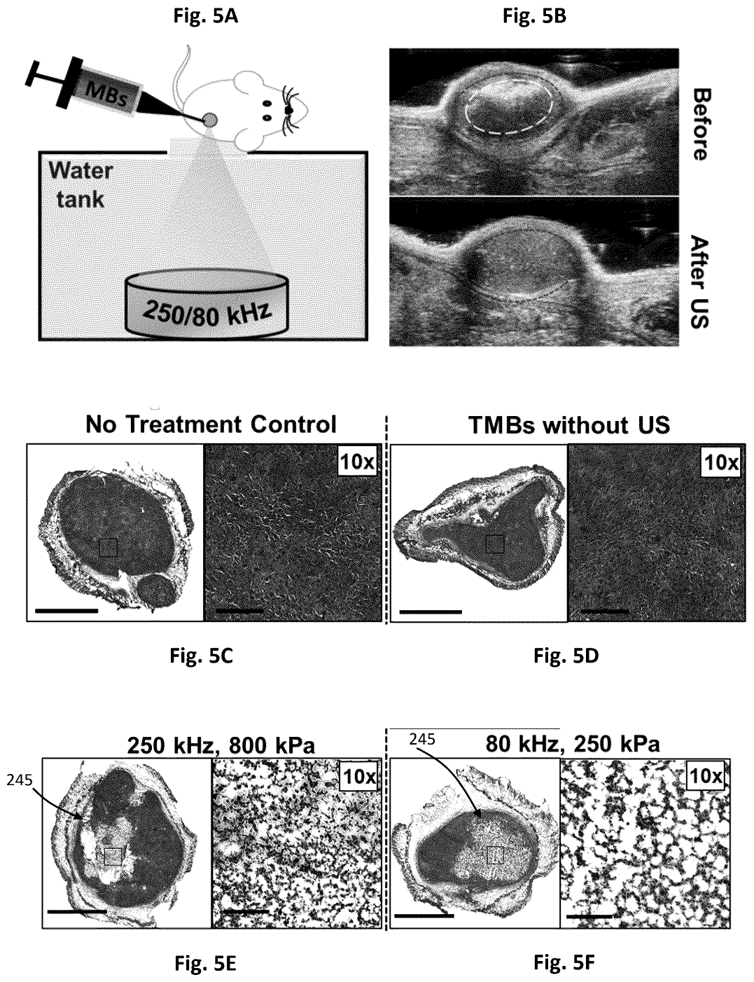

Reference is made to FIGS. 5A-5F, which show the results of an in vivo assay musing a mouse model for treating breast cancer with a combined MBs and low frequency US treatment.

For the in vivo assay, Female FVB/NHan®Hsd mice (8 to 12 weeks old, 20-25 g, Envigo, Jerusalem, Israel) were used as breast cancer animal models for evaluation. MET1 cell line, a mouse mammary tumor line, was cultured in Dulbecco modified Eagle medium (DMEM, high glucose, supplemented with 10% v/v fetal bovine serum, 1% v/v penicillin-streptomycin, 0.292 g/L L-glutamine and 0.11g/L sodium pyruvate) at 37° C. in a humidified 5% CO2 incubator until about 85% confluency on the day of the injection. Cells were then collected via dissociation with TrypLE™ Express and resuspended at 1×10⁶ cells in 25 μL PBS+/+ for bilateral subcutaneous injection into #4 and #9 inguinal mammary fat pad to obtain primary tumor model. Tumor size was recorded every 4 days until they reached approximatively 4 mm in diameter (approximately 14 days after cell injections). All animal procedures were performed according to guidelines of the Institutional Animal Research Ethical Committee.

Reference is made to FIG. 5A, which shows the experimental setup 200 used to perform the in vivo assay. A total of 28 bilateral FVB/NHan®Hsd tumor-bearing mice were studied. A 250 kHz/80 kHz spherically-focused single-element transducer 202 was placed at the bottom of a degassed water tank 204 facing upwards and aligned to focus at an agar spacer 206 which positioned the tumor 208 at the focal depth of the transducer (z=45 mm). Before ablation treatment, 2×10⁷ TMBs (prepared as described herein above) in 20 μL degassed PBS solution were injected intratumorally (IT) so that TMBs 209 would be located within tumor 208. The TMB solution was freshly prepared before each IT injection. US gel was used, and the treated area was shaved and fur further removed using a depilatory cream for a better sonic coupling. Mouse 210 was positioned on its side, on top of the agar spacer. Anesthesia was induced with 2% isoflurane in ambient air (180 mL/min). The agar spacer was prepared as previously described for the agar cube.

Once the mouse was anesthetized and in position, transducer 202 was activated to apply focused US at TMBs 209 located within tumor 208. For the 250 kHz center frequency treatments, the PNP of the US was 800 kPa (MI of 1.6). For the 80 kHz center frequency treatments, the PNP of the US was 250 kPa (MI of 0.9). The US parameters were chosen so that the CI for both frequencies remained similar (˜3.2), while the MI remained below 1.9, which is the FDA-mandated upper limit for safety. For both frequencies, each US pulse contained 125-cycles of a sinusoid US signal at the determined frequency, and each 125-cycle US pulse was repeated at a pulse repetition frequency of 30 Hz, for a total US treatment duration of 1 minute.

The TMBs tumor-distribution before and after treatment was assessed by US imaging using the Vevo 2100 Ultrasound system (not shown). Control groups included non-treated controls (NTC), TMBs only (without US treatment) and US only. Bilateral tumor-bearing mice were sacrificed one day after US-mediated ablation for tumor removal and histological analysis (see FIGS. 5C-5F). The treated tumors were dissected out and cryo-sectioned to 12 um thick slices, which were then stained with hematoxylin (Leica 3801542) and eosin (Leica 3801602) (H&E) according to standard procedure. The H&E slides were scanned using the Aperio Versa 200 slide scanner (Leica Biosystems, Buffalo Grove, IL; not shown) at 20× optical magnification.

Low-frequency TMB oscillations impact on breast cancer tumors was evaluated in vivo on bilateral breast cancer tumor-bearing mice, to compare the impact of 250 kHz vs. 80 kHz center frequency. US was applied to the tumors following an intratumoral (IT) injection of TMBs. The PNP for the 250 kHz (800 kPa, MI=1.6) and for the 80 kHz (250 kPa, MI=0.9), were chosen to maintain a constant cavitation index of ≈3.2, while operating below the FDA MI upper limit of 1.9. In both frequency regimes, all of the TMBs dissipated due to cavitation following exposure to the applied US. As shown in the representative image provided in FIG. 5B, US imaging before and after treatment confirmed complete TMB destruction following insonation.

Reference is made to FIGS. 5C-5F, which show examples of histological evaluation performed 24 hours post US treatment. FIG. 5C shows representative sample of a cryosectioned tumor from a non-treated control tumor that was not administered the TMBs and was not exposed to US treatment. FIG. 5D shows a representative sample of a cryosectioned tumor from a control tumor that was administered the TMBs but did not undergo US treatment. FIG. 5E shows a representative sample of a cryosectioned tumor from an experimental tumor that was administered the TMBs, then treated with focused US at 250 kHz and 800 kPa. FIG. 5F shows a representative sample of a cryosectioned tumor from an experimental tumor that was administered the TMBs, then treated with focused US at 80 kHz and 250 kPa. For each of FIGS. 5C-5F, the image on the left shows an entire cryosection of a tumor, and the image on the right shows a 10× magnification of a region in the tumor. In FIGS. 5C-5D, the 10D× magnified image is from within a region that received TMBs. In FIGS. 5E-5F, the 10× magnified image is from within a treatment region that received TMBs and was exposed to the focused US.

The in vivo study described above confirmed the effectiveness of the combined TMB and focused US treatment in ablating tumors. FIGS. 5E-5F show the presence of defined lesions 245 with an average diameter of ˜2.5 mm in tumors receiving the combined treatment. By contrast, no lesions were visible in the control groups, as exemplified in FIGS. 5C-5D.

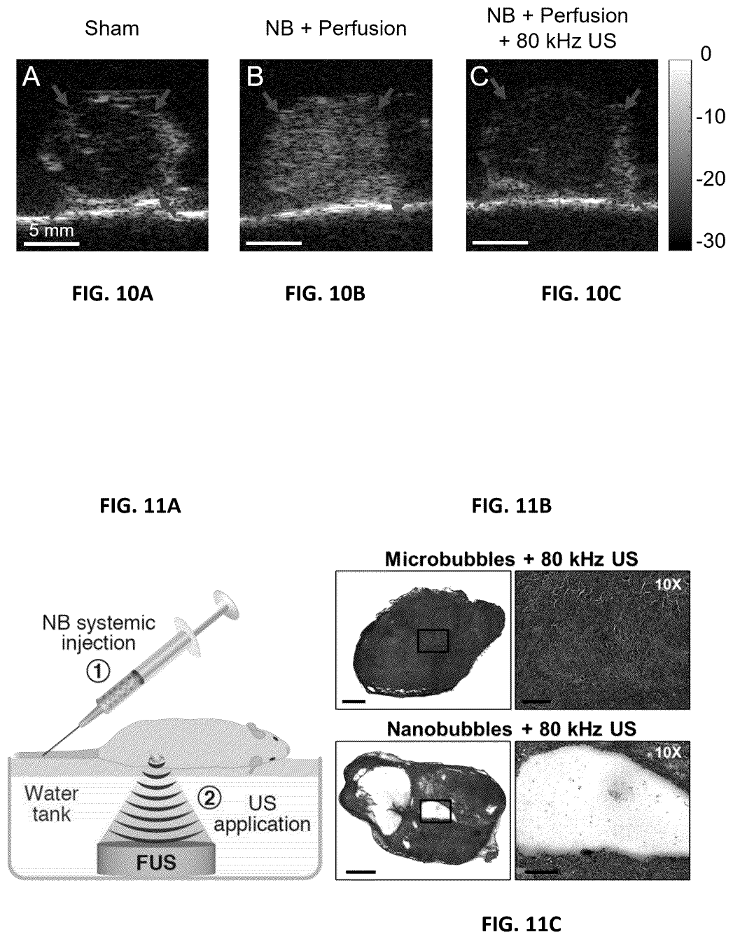

The study also shows that the 80 kHz US treatment was more effective in tumor ablation than the 250 kHz US treatment. As shown in FIGS. 5E-5F, 10×-magnified images of the lesion region indicate a larger degree of tissue perforation with the 80 kHz treatment region compared to the 250 kHz treatment region: Quantification of total white area (TWA) in the magnified lesion images, which correspond to the holes, resulted in an average of 48.6±6.8% TWA for the 80 kHz, compared to 31.3±3.8% TWA for the 250 kHz (p<0.05).

As noted above, the MI of the 80 kHz treatment was 0.9, which is substantially lower that the MI of the 250 kHz treatment, which was 1.6. In addition, the CI of the two US treatment regimens were the same. Despite the lower MI, LE bubble histotripsy exemplified by 80 KHz US treatment was surprisingly found to be more effective in inducing tumor ablation compared to standard low power bubble histotripsy exemplified by 250 kHz US treatment, even under US parameters. This feature of LE bubble histotripsy provides an advantageous effect in lower likelihood of tissue damage in off-target regions lacking in MBs.

Aside from the enhanced MB vibrational response, the use of low frequency US enhances penetration depth because of the reduced tissue absorbance at this frequency range that minimizes attenuation compared to higher frequencies. Thus, LE bubble histotripsy better allows for safer ablation of deeper tumors without risking off-target tissue damage. Further, the low frequency enlarges the focal zone which advantageously aids in treating larger volume simultaneously.

### Example 5

### Nanobubbles Preparation and Characterization

NB synthesis was performed based on Perera R et. al, Nanoscale. 2019; 11 (33):15647-15658. Briefly, 1,2-dibehenoyl-sn-glycero-3-phosphocholine (C22), 1,2-dipalmitoyl-sn-glycero-3-phosphate (DPPA), 1,2-dipalmitoyl-sn-glycero-3-phosphoethanolamine (DPPE), and 1,2-distearoyl-sn-glycero-3-phosphoethanolamine-N-[methoxy(polyethylene glycol)-2000] (ammonium salt) (DSPE-mPEG 2000) (Sigma-Aldrich) were dissolved into propylene glycol by heating at 80° C. and sonicating. Glycerol was mixed to phosphate buffer solution (PBS) and the mixture was preheated to 80° C. before addition to the lipid solution. The lipids were mixed to a molar ratio of 18.8:4.2:8.1:1 and a final lipid concentration of 10 mg/mL. The resulting mixture was then sonicated at room temperature for 10 min. 1 mL of the resulting lipid mixture was transferred to a 3 mL headspace vial. Vials were saturated with octafluoropropane (C₃F₈) gas, then capped with a rubber septum and sealed with an aluminum seal. The vials were stored at 4° C. until usage. Immediately prior to each experiment, a vial was activated by mechanical shaking for 45 sec with a Vialmix shaker (Bristol-Myers Squibb Medical Imaging Inc., N. Billerica, MA). The vial was placed inverted in a centrifuge (5810R centrifuge, Eppendorf AG, Hamburg, Germany), and then centrifuged at 50 rcf for 5 min. 200 μL of the NBs solution was pulled out of the inverted vial with a 21 G needle, at a distance of 5 mm from the bottom of the vial. MBs were prepared as reported previously using a thin film hydration method. A particle sizing system (AccuSizer FX-Nano, Particle Sizing Systems, Entegris, MA, USA) was used to measure the size and concentration of the purified MBs and NBs. The bubbles were used within 3 h of their preparation. The size distribution and concentration varied by less than 10% between the measurements.

The TEM experiments used to visualize the NBs morphology used a TEM (JEM-1400Plus, JEOL, Tokyo, Japan) that was operated at 120 kV. Briefly, 5 μL of the NBs sample were pipetted onto glow-discharged carbon grids. After 30 sec of incubation, the sample was washed with buffer. 5 μL of 1% uranyl acetate were added for 30 sec, then removed, and left to air-dry. The grids were then imaged.

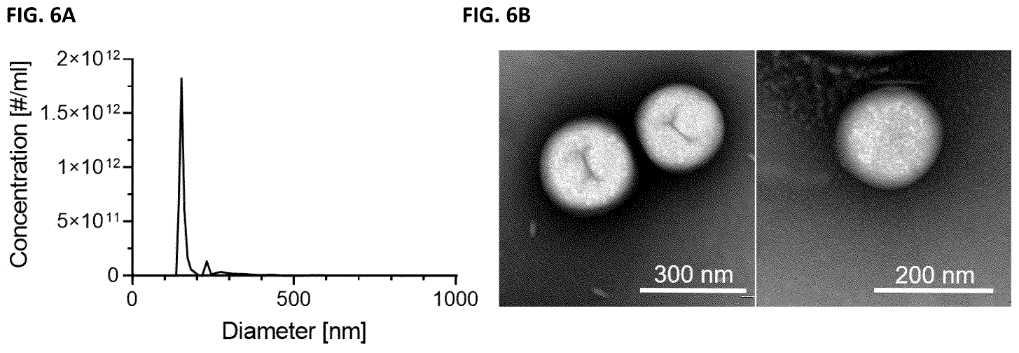

The size distribution and concentration of freshly prepared NBs were measured using a particle sizing system (FIG. 6A). Mean NB diameter was 170±60 nm, and the concentration was 3.3×1012 particles/ml. The same system was used to measure MB size and concentration. Mean MB diameter was 1.67±0.97 μm and the MBs concentration was 1.79×1010 particles/ml. Morphology of NBs was then characterized using transmission election microscopy (TEM). NBs exhibit a spherical morphology with diameters that match the mean diameter measured with the particle sizing system (FIG. 6B).

### Example 6

### Characterization of Low Frequency Nanobubbles Insonation-Low Frequency Ultrasound Setup

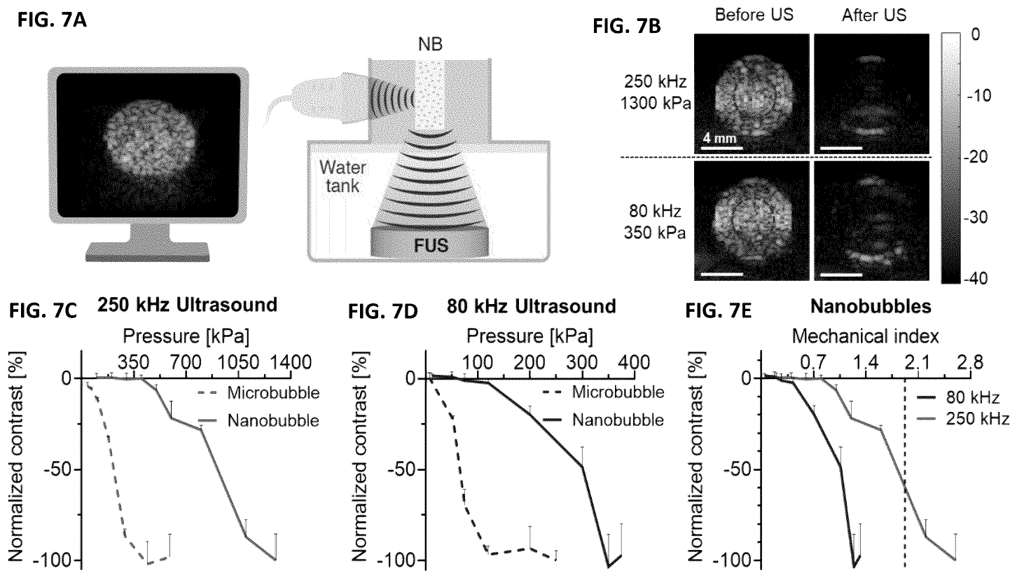

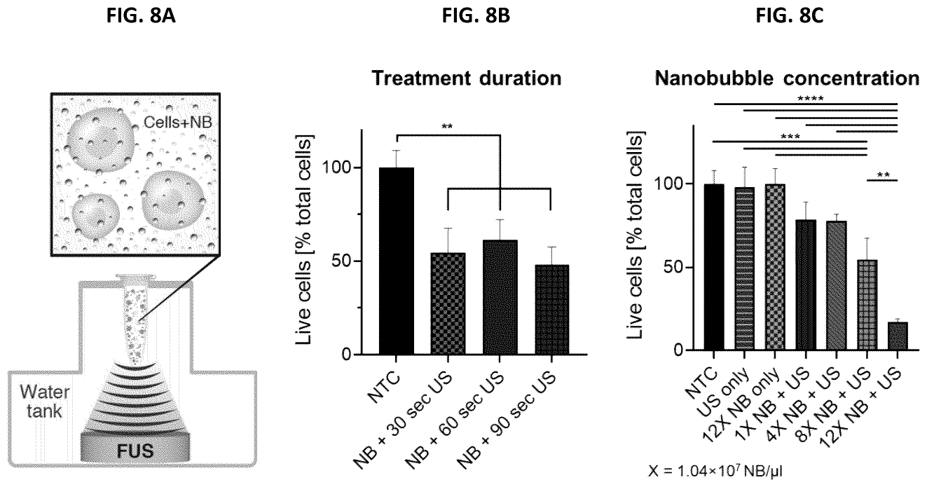

The US setup was as described above, and as further illustrated in FIGS. 7A, 8A. The setup is composed of a water tank, where a spherically focused single-element transducer (H115, Sonic Concepts, Bothell, WA, USA) was placed on its bottom facing upwards. The transducer was focused to a distance of 45 mm. The H115 transducer supports both 250 and 80 kHz center frequencies via custom matching networks (purchased from Sonic Concept). The transducer transmitted a sinusoid at the desired frequency. The waveform was generated using a transducer power output unit combining an arbitrary waveform generator together with a radiofrequency amplifier (TPO-200, Sonic Concepts). At 80 kHz, approximatively a third of maximal pressure is obtained compared to the pressure obtained with 250 kHz center frequency. Calibration measurements of the transmitted pressure the were performed using a calibrated needle hydrophone (NH0500, Precision Acoustics, UK). For each experiment, the desired target was placed at the focal spot of the transducer. For the NB characterization experiments in tissue mimicking phantoms, an agarose phantom containing an inclusion filled with NBs suspension was used. For the in vitro experiments, a 0.5 mL Eppendorf tube containing a mixture of breast cancer cells and NBs was used. For the in vivo experiments, the mouse was positioned such that the breast cancer tumor was located at the focal spot.

Agar phantom was prepared by dissolving agarose powder (A10752, Alfa Aesar, MA, USA) in distilled water to a 1.5% solution, followed by heating to completely dissolve the agar powder. The solution was poured into a mold containing a 6 mm rod inclusion and cooled at ambient temperature. In each experiment, a mixture of 3.75×10⁹ NBs diluted in 300 μl degassed phosphate buffered saline (PBS) was injected into the rod inclusion in the agarose mold. For the MB experiments, 3×10⁶ MBs diluted in in 300 μl degassed PBS was used. Experiments were performed in a dual imaging-therapy setup, where an L7-4 imaging transducer (Philips ATL) was placed perpendicularly to the therapeutic transducer (FIG. 7A) and controlled by a programmable US system (Verasonics, Vantage 256, Verasonics Inc., Redmond, WA, USA). Images of the NB-filled Inclusion were acquired by the imaging transducer before and after the application of the low frequency therapeutic US. Low frequency insonation consisted of a sinusoid US signal with a burst length of 1.56 ms and a pulse repetition frequency (PRF) of 30 Hz. The contrast before and after the application of low frequency US was calculated by using formula 2:

$$Contrast [ dB ] = 20 {log}_{10} ( \frac{{u}_{i}}{{u}_{o}} ) \qquad ( 1 )$$

where μᵢ and μₒ are the mean of an area inside the NB inclusion before and after the application of low frequency US, respectively. Locations used for contrast calculations were marked by red circles (FIG. 7B).

The tissue mimicking experiments were aimed to compare the NBs contrast reduction following insonation with two low frequencies of 250 and 80 kHz, as a mean to evaluate the NBs acoustical response. In addition, for each frequency, the acoustical behavior of MBs and NBs was directly compared. A dual imaging-therapy setup was used to assess the impact of insonation parameters on the contrast of a NB filled inclusion (FIG. 7A). This was performed by imaging the inclusion with an imaging transducer before and after the application of a 1 second low frequency US at either 250 or 80 kHz (FIG. 7B). Inertial cavitation and fragmentation resulting from the NBs oscillations is expected to reduce the contrast due to the reduced number of intact bubbles in the inclusion. Thus, analysis of the inclusion contrast reduction as a function of the insonation parameters serves as an indicator of the NBs cavitation status. Maximal contrast reduction is desired in order to maximize NB-mediated mechanotherapy.

Initially, the NBs concentration was optimized. At low NB concentrations, inclusion contrast remains low. Increasing the NB concentration increases the contrast up to a point where the US signal reaches a peak and begins to decrease due to high concentration that blocks the propagation of the US beam. The peak contrast was reached at a NB concentration of 1.25×10¹⁰ NBs/mL, which was chosen for the tissue mimicking phantom experiments. Next, NB solution at the optimal concentration was placed in the inclusion, followed by application of low frequency US (either 250 or 80 kHz) with different PNPs. For each center frequency, contrast reduction was evaluated for MBs or NBs. Higher PNPs were required in order to achieve maximal contrast reduction for NBs, as compared to MBs, for the two frequencies tested (FIG. 7C and FIG. 7D). For the 250 KHz frequency treatment, maximal contrast reduction of MBs was obtained at a PNP of 440 kPa (MI of 0.9), whereas a PNP of 1300 kPa (MI of 2.6) was required for the NBs complete destruction (FIG. 7C). For the 80 kHz insonation, maximal contrast reduction of MBs was obtained at a PNP of 120 kPa (MI of 0.4), whereas NBs compete destruction required a PNP of 350 kPa (MI of 1.2, FIG. 7D). Direct comparison of the contrast reduction of NBs as a function of the MI for 250 and 80 kHz indicate that NB destruction occurs at lower MI for the 80 kHz center frequency (FIG. 7E). Notably, in order to remain below an MI of 1.9, these results suggest that 80 kHz insonation is required. Therefore, in the following experiments, 80 kHz US was used.

### Example 7

### In Vitro Nanobubble-Mediated Low Frequency Insonation of Breast Cancer Cells

4T1 cells, metastatic triple negative murine breast carcinoma cell line was purchased from ATCC. 4T1 cells were cultured in RPMI 1640 (10% v/v fetal bovine serum, 1% v/v penicillin-streptomycin, and 0.292 g/L L-glutamine). Cells cultures were incubated at 37° C. with in a humidified 5% CO₂ incubator. About 85% cells confluency was reached on the day of each experiment. Cells collection was performed using TrypLE Express dissociation reagent (Gibco Corp, 12604-013, Grand Island, NY, USA). Cells were then suspended at a concentration of 3.3×10⁶ cells/mL in degassed PBS containing calcium and magnesium (PBS+/+).

A mixture of 2×10⁵ cells and NBs (at different tested concentrations, as described below) was then transferred into 0.5 mL Eppendorf tubes and degassed PBS+/+ was added to a final volume of 0.48 mL. Each Eppendorf tube was positioned at the focal spot of the low frequency US setup, and 80 kHz US with an MI of 1.3 (PNP of 375 kPa), PRF of 30 Hz and 1.56 ms burst length was applied to the tube.

Optimization experiments of the effect of total treatment duration and the NB concentration on cell viability were then performed. Treatment duration tested were 30, 60, and 180 sec, for a constant NB concentration of 8.32×10⁷ NBs/μl. For the NB concentration optimization experiments, a constant treatment duration of 30 sec was used. The NBs concentrations tested were 1.04×10⁷ NBs/μL, 4.12×10⁷ NBs/μL, 8.32×10⁷ NBs/μL and 12.5×10⁸ NBs/μL. Control groups included NTC, US treatment only and NB only (using the highest NB concentration of 12.5×10⁷ NBs/μL). After each US treatment, cells were grown for 72 hours in six-well tissue culture dishes containing complete medium supplemented with 2.5% v/v penicillin-streptomycin. 72 hours post US treatment, cells viability was assessed. Cells were collected in 500 μL of TrypLE Express. Cell Drop (DeNovix Inc., Wilmington, USA) and 0.4% Trypan Blue (Sigma-Aldrich) in a 1:1 ratio with the cells suspension were used for live cell counting. All treatments were analyzed in triplicate.

In vitro experiments were aimed to assess the impact of NB low frequency insonation (80 kHz) on cancer cell viability as a function of treatment duration and NBs concentration. Insonation was applied to Eppendorf tubes containing a mixture of NBs and breast cancer cells (FIG. 8A). Experiments were conducted at an MI of 1.3. Initially, the effect of treatment duration on cell viability was assessed for a constant concentration of NBs (8.32×10⁷ NBs/μL). No significant difference was found between the different treatment durations tested (30, 60, and 90 sec) (FIG. 8B). Therefore, in order to minimize US exposure, a treatment duration of 30 sec was chosen for the following experiments. Next, the NBs concentration was optimized. The initial concentration was x=1.04×10⁷ NBs/μL, and 3 additional concentrations of 4, 8 and 12 times the initial concentration were tested. Control groups included no treatment control (NTC), US only and NBs only (at the highest NB concentration of 12×). All control groups yield similar cell viability of 100%. Concentrations of 1× and 4× reduced viability to 78.3±7% (not significant compared to control groups, p>0.05). 8× concentration reduced viability to 54.6±12.9% (p<0.001 compared to control groups), while 12× concentration reduced viability to 17.3±1.7% of live cells (p<0.0001 compared to control groups) (FIG. 8C).

### Example 8

### In-Vivo NB Tumor Distribution Using Contrast Harmonic Ultrasound Imaging

In all of the in vivo experiments, tumor bearing mice were anesthetized with 2% isoflurane using a low flow vaporizer system (SomnoFlo, Kent Scientific). The tumor area was completely shaved and US gel was applied. The mice were positioned on their side, on top of an agarose pad, such that the tumor was located at the focal spot of the transducer (FIG. 11A). For the NB tumor distribution imaging experiments, a high frequency US transducer (L22-8v, Verasonics, USA), controlled by a programmable US system (Verasonics, Vantage 256, Verasonics Inc., Redmond, WA, USA) was used to acquire the US images. A Contrast pulse sequencing (CPS) mode with coherent compounding was implemented by sending 3 successive single cycle pulses (+½, −1, +½). In addition, coherent compounding was achieved by transmitting plane waves at 3 different angles (−5°, 0°, 5°) and one full frame was the combination of the 9 transmit/receive events. The transmitted center frequency was 10 MHz. Baseline of the tumor core signal was acquired prior NBs injection. 6.6×10¹¹ NBs in 200 μl of PBS were then systemically injected and the tumor was imaged periodically for 10 min post-injection.

For assessment of NB extravasation and accumulation within the tumor tissue, 10 min post NBs injection anesthetized mice were euthanized. Cardiac perfusion was performed with 15 ml of PBS through the left ventricle. After perfusion, the tumors were extracted and US imaging was performed to detect US signal produced from the NBs that were accumulated within the tumor tissue. The extracted tumors were then insonated with 80 kHz center frequency US, using an MI of 1.3, a burst length of 1.56 ms, a PRF of 30 Hz, and a total treatment duration of 2 minutes. After US treatment, US imaging was performed using the mentioned above CPS sequence to evaluate the US signal within the tumor. Sham control tumors underwent cardiac perfusion without NBs injection, followed by US imaging of the perfused tumors.

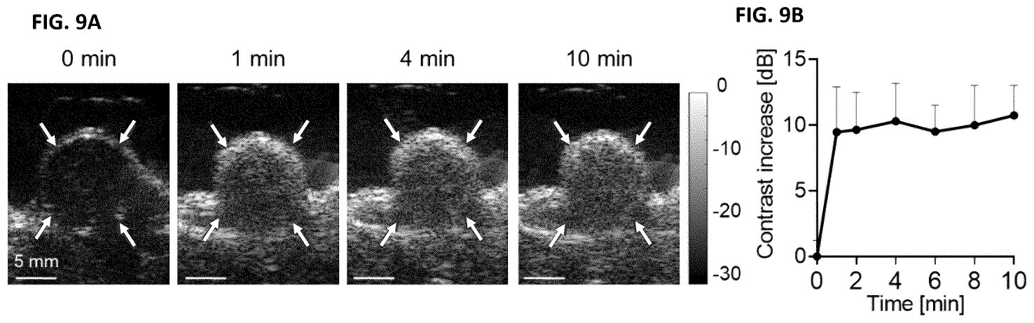

Since NBs are a theranostic agent, their distribution within the tumor was assessed via contrast-enhanced US imaging, following a systemic NBs injection in breast cancer tumor bearing mice (FIGS. 9A-B). Prior to NB injection, tumor was dark and anechoic (FIG. 9A, 0 min). After NB injection the tumor became hyperechoic. The increase in tumor contrast as a function of time post injection of NBs resulted in a contrast increase by 9.5±3.4 dB at 1 min. The contrast remained similar for 10 min following NB administration (FIG. 9B).

Tumor contrast enhancement following NB administration is a combination of the echoes from NBs circulating within the tumor blood vessels, and the NBs that were able to extravasate into the tumor tissue as a result of the EPR effect. In order to confirm the presence of tumor-accumulated NBs, cardiac perfusion performed 10 min post NB injection was used to wash the NBs within the blood vessels. Tumors were collected and imaged via contrast harmonic US imaging. Tumor cores in the sham groups remained anechoic, whereas an increase of 10.3±2.5 dB in contrast was detected in the NB +perfusion group (p<0.05, FIGS. 10A-10B).

80 kHz US was then applied to the collected perfused tumors (MI of 1.3) in order to implode the tumor-accumulated NBs, and confirm that the US signal in the tumors following perfusion arises from the NB accumulation. Contrast harmonic US imaging of the perfused tumors following the application of low frequency US showed a reduction in tumor contrast by 8.3±1.0 dB, compared to the same tumors before the application of low frequency US (p<0.05, FIGS. 10B-10C).

### Example 9

### In Vivo Nanobubble-Mediated Low Frequency Insonation of Breast Cancer Tumors

### Breast Cancer Animal Model

A total of 37 bilateral FVB/NHanHsd tumor-bearing mice were used for the in vivo studies. Met-1 mouse breast carcinoma cells were injected into 8 to 12 weeks old female FVB/NHanHsd mice (Envigo, Jerusalem, Israel). Cells were cultured at 37° C. in a humidified 5% CO₂ incubator in Dulbecco modified Eagle medium (DMEM, high glucose, supplemented with 10% v/v fetal bovine serum, 1% v/v penicillin-streptomycin and 0.11 g/L sodium pyruvate). During the day of the injection, Met-1 cells were collected with TrypLE Express dissociation reagent to a final concentration of 1×10⁶ cells in 25 μL PBS+/+. Cells were subcutaneously injected into #4 and #9 inguinal mammary fat pad. Tumor size was recorded every 4 days until they reached approximatively 4 mm in diameter. Met-1 mouse breast carcinoma cells were a gift from Prof. Jeffrey Pollard, University of Edinburgh, Edinburgh, UK, and Prof. Neta Erez, Tel Aviv University, Tel Aviv, Israel. All animal procedures were performed according to guidelines of the Institutional Animal Research Ethical Committee.

6.6×10¹¹ NBs in 200 μl or a volume of 50 μl containing 2×10⁷ MBs were systemically injected. 10 minutes post injection, 80 kHz US was applied to the tumor, using an MI of 1.3, burst length of 1.56 ms, a PRF of 30 Hz, and a total treatment duration of 2 minutes. Additional control groups included NTC, and US only. Mice were sacrificed 24 hours after treatment for tumor extraction and histology analysis. For histology, tumors were cryo-sectioned to 12-μm-thick slices and stained with hematoxylin (Leica 3801542) and eosin (Leica 3801602) (H&E) according to a standard procedure. The slides were then scanned with the Aperio Versa 200 slide scanner (Leica Biosystems, Buffalo Grove, IL) at 20× optical magnification.

NB-mediated low frequency US insonation of tumors was performed in vivo. Ten minutes post systemic injection of NBs, 80 kHz US with an MI of 1.3 was applied to the breast cancer tumors (FIG. 11A). Control groups included NTC, only US and mice that underwent the same treatment however were systemically injected with MBs instead of NBs. Twenty-four hours post treatment, tumors were collected for histological evaluation. Tumors that were treated with MBs+80 KHz US, yield similar pathology as the NTC group where no damage was observed on histology (FIG. 11B). Tumors treated with NBs+80 kHz US demonstrated extensive tumor damage with visibly defined lesions and perforated tumor tissue (FIG. 11C).

### Example 10

### Nanodroplet (ND) Preparation and Characterization

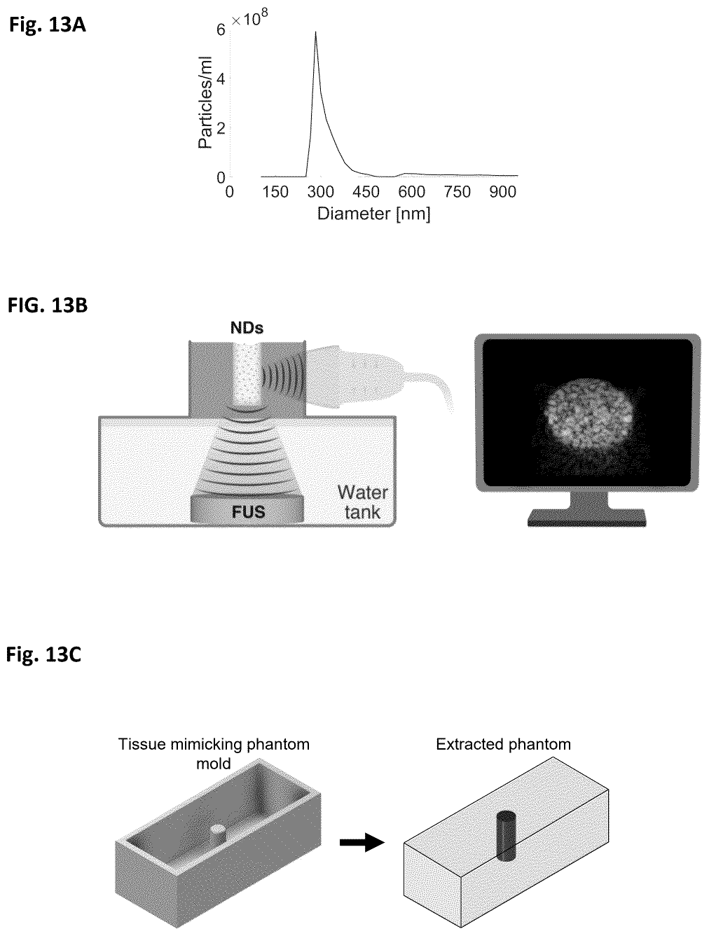

The ND preparation included two stages: First, the MBs precursor solution and activation were prepared as described previously (Ilovitsh T, et. al., (2018) Scientific Reports Nature Publishing Group, 2018; 8) and aboveherein. Briefly, the lipids disteroylphosphatidylcholine (DSPC) and 1,2-distearoyl-sn-glycero-3-phosphoethanolamine-N-[meth-oxy(polyethyleneglycol)-2000] (ammonium salt) (DSPE-PEG2K) (Avanti Polar Lipids, Alabaster, AL) (1 mg per 1 mL) were combined at a molar ratio of 90:10 mol/mol and prepared using a thin film hydration method. A buffer mixture of glycerol, propylene glycol, and PBS (pH 7.4) with a volume ratio of (16:3:1) was added to the lipids and sonicated at 62° C. The precursor solution was aliquoted into vials with a liquid volume of 1 mL and saturated with perfluorobutane. MBs were formed via standard agitation techniques using a vial shaker. In the second stage, a condensation procedure was performed to phase change the MBs into NDs, as described in Sheeran et. al., 2011, Langmuir 2011; 27:10412-10420. The MB vials were immersed in an isopropanol bath at a temperature between −10° C. and −13° C. and swirled gently for approximately 2 min. A 25 G syringe needle containing 50 mL of perfluorobutane gas was then inserted into the vial septum and the plunger was depressed slowly until the process of condensation was observed. The size distribution and concentration of the NDs were measured with a particle counter system (AccuSizer FX-Nano, Particle Sizing Systems, Entegris, MA, USA) and the results are presented in the graph shown in FIG. 13A, demonstrating an average diameter of about 330 nm. The NDs were stored at 4° C. during the experiments. The NDs were used within 3 hours of their preparation.

### Example 11

### Theoretical Prediction of Microbubble Expansion

Standard MBs were fabricated as described above. The theoretical predictions for the MB expansion ratio were simulated using the Marmottant model implemented in MATLAB. Parameters such as MB composition, US excitation wave and the MBs' surrounding medium viscosity and density were taken into consideration in this model. The effects of 3 center frequencies (850, 250 and 80 kHz) and the PNP (between 0-1000 kPa) on MB expansion ratio were evaluated. The parameters were identical to those in (Ilovitsh et al. 2018). The surface tension of the MB outer radius was set to 0.073 N/m (saline) and to 0.04 N/m for the inner radius. The shell density was 1000 kg/m3, the shell shear modulus was 122 MPa, the shell viscosity was 2.5 Pa's, the shell surface dilatational viscosity was 7.2×109 N, and the elastic compression modulus was 0.55 N/m. The shell thickness was set to 1.5 nm. The initial MB radius value was 0.75 μm.

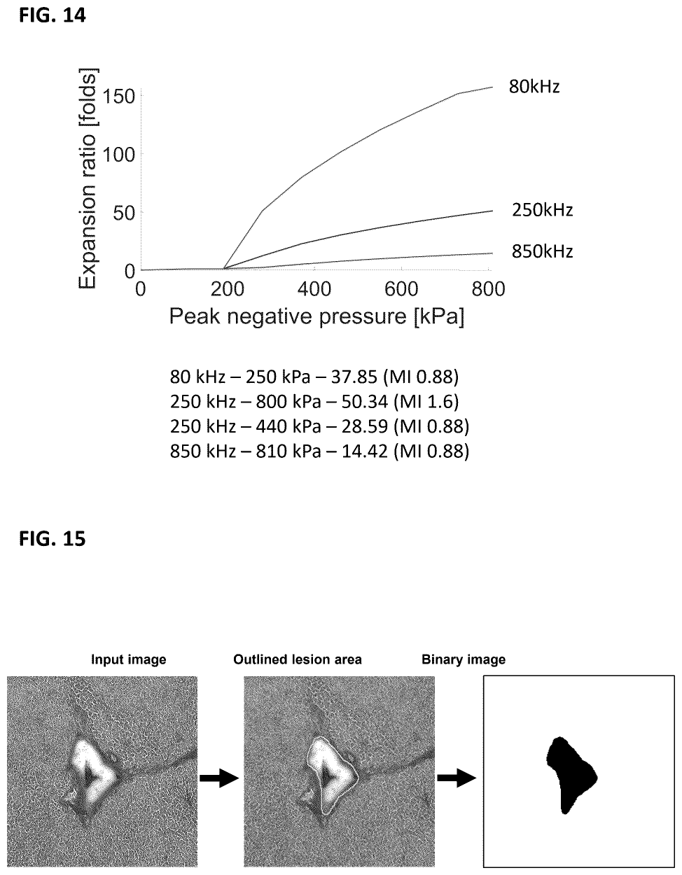

After the vaporization of NDs into gaseous MBs, as detailed below, low frequency US was applied to implode the MBs. As an initial step, theoretical predictions using the Marmottant model were made for the three center frequencies used in the experiments (850, 250, and 80 kHz). The MB expansion ratio, defined as the maximal diameter divided by the resting diameter, was calculated for each frequency as a function of the PNP. The results are presented in FIG. 14. At a PNP of 810 kPa, the predicted expansion ratio was 14, 51 and 157 for 850, 250 and 80 kHz, respectively. When working with the same MI of 0.9, the predicted expansion ratio was 14, 29 and 38 for center frequencies of 850, 250, and 80 kHz, respectively. Since the MB transition to inertial cavitation at expansion ratios above 3.5, it was expected that when working at an MI of 0.9, the MBs would fragment at each of the three low frequencies. However, the expansion was predicted to be the highest for the 80 kHz center frequency.

### Example 12

### 1D Ultrasound Setup Experimental System

To perform the two-step method of converting ND to MB (using high frequency US) and the subsequent imploding of MBs (using low frequency US), a dual imaging-therapy experimental setup/system was used. The dual imaging system includes a water tank, and two perpendicularly aligned transducers that insonified a sample located at the focus of both transducers (as illustrated in FIG. 13B)). The first transducer includes an imaging transducer (L7-4, Philips, ATL), controlled by a programmable US system (Vantage 256, Verasonics Inc., Redmond, WA, USA). This transducer has 128 elements, with an element size of 7 mm×0.283 mm (height×width), a kerf width of 0.025 mm and operates at a center frequency of 5 MHz. This transducer was used for the ND vaporization process by transmitting a 2-cycle pulse focused on the location of the injected NDs (distance of z=13 mm). The imaging transducer was also used for acquiring US images of the NDs before and after each optimization experiment. The second transducer was a spherically focused single-element therapeutic transducer (H117, Sonic Concepts, Bothell, WA, USA) that was located at the bottom of the water tank. This transducer is capable of operating at center frequencies of 850, 250 and 80 kHz using custom matching networks. The transducer focus for all three frequencies was at a distance of 45 mm. The transducer was operated using a transducer power output unit (TPO-200, Sonic Concepts, Bothell, WA, USA). Both transducers PNPs were calibrated with a needle hydrophone (NH0200, Precision Acoustics, UK) in situ. In each experiment, an agarose phantom was placed at the focal spot of both the imaging and therapeutic transducers and contained either a diluted ND solution or the ex-vivo chicken tissues samples (such as, liver or breast sample).

Agarose phantom preparation—All experiments were performed using an agarose mold that was located at the focal spots of both transducers in the dual imaging-therapy setup. The phantoms were prepared by mixing 1.5% agarose powder (A10752, Alfa Caesar, MA, USA) and deionized water. The solution was heated until all the powder was completely dissolved, and then was poured into a custom mold and allowed to cool. This 3D printed mold measured 65 mm×25 mm×20 mm (length×width×height) with a 15 mm height rod at its center, as shown in FIG. 13C. For the ND activation and detonation optimization experiments, a mold with a 6 mm rod was used, and for the ex-vivo experiments a mold with an 8 mm rod was used. Once the solution congealed, it was extracted from the mold. Therefore, the agarose phantom has a negative shape to that of the mold, with a rod-inclusion that ends with a 5 mm base of agarose that prevents the leakage of interior of the well-shaped inclusion (FIG. 13C).

### Example 13

### Nanodroplet Vaporization Optimization Experiments

These experiments were designed to optimize the vaporization process of NDs into gaseous MBs. The imaging transducer had a frequency of 5 MHz, and utilized two-way focusing for the activation process and for the US images acquisition. In each experiment, a mixture of 0.0132−3×10⁷ NDs/mL diluted in 300 μl degassed phosphate buffered saline (PBS) was injected into the rod inclusion in the agarose mold, and filled the inclusion completely. The imaging transducer captured an image of the NDs before activation. Next, a 2 cycle excitation pulse, with a pulse repetition frequency (PRF) of 20 Hz and a total duration of 2-10 seconds and PNPs ranging 2.3-4.5 MPa (MI of 1-2) was applied to the NDs inclusion to vaporize the NDs into MBs. Immediately after vaporization, the imaging transducer acquired another US image. Post-processing of the captured images was used to calculate the change in contrast before and after the vaporization process, using formula 2.

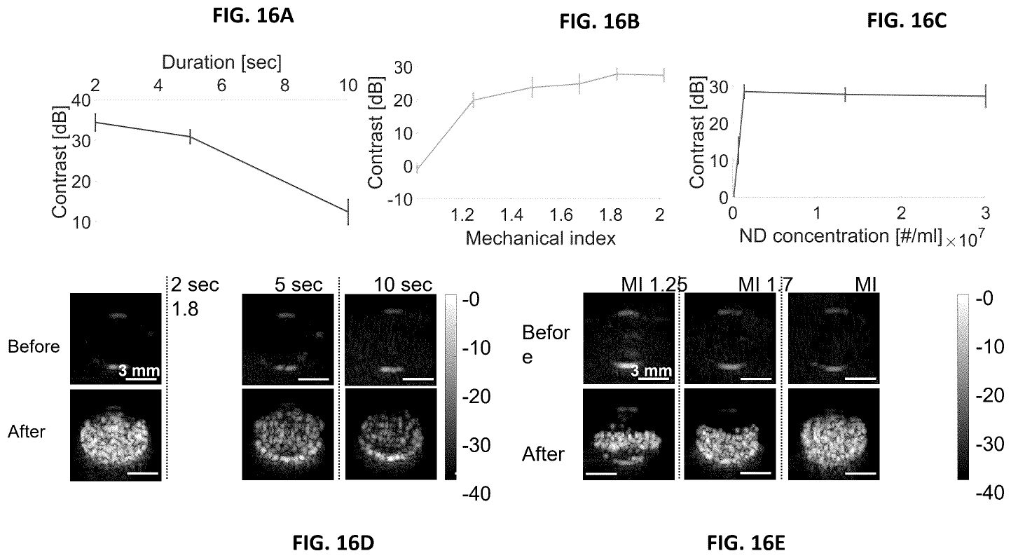

NDs with a mean diameter of 300 nm were fabricated as detailed above. Optimization of the ND vaporization process into gaseous MBs was implemented by the dual imaging-therapy setup (as illustrated in FIG. 13B). Vaporization was employed by the L7-4 imaging transducer at a center frequency of 5 MHz. The activation duration (FIG. 16A), the applied PNP (FIG. 16B), and the ND concentration (FIG. 16C) were optimized. US imaging was used to capture US image(s) prior to vaporization while the ND inclusion appears dark, and after vaporization, where the inclusion becomes hyperechoic as a result of the MB generation. The resulting contrast for each parameter was analyzed. An activation duration of 2 seconds yielded the highest contrast of 34.3 dB, and thus was used in further experiments ((FIG. 16A). Exemplary images of before and after activation are presented in FIG. 16D. The contrast increased when increasing the activation pulse MI (FIG. 16B); however, the slope moderated beyond an MI of 1.2. In order to maximize the contrast while operating below an MI of 1.9, an MI of 1.8, which yielded a contrast of 27 dB was selected (examples of before and after images are presented in FIG. 16E. The contrast increased as the ND concentration increased, however above a concentration of 0.132×10⁷ the contrast did not change significantly (FIG. 16C). Accordingly, a value of about 1.32×10⁷ NDs/mL was used in further experiments.

### Example 14

### Low Frequency MB Insonation Optimization Experiments

In each experiment, a mixture of 1.32×10⁷ NDs/mL diluted in 300 μl of degassed PBS was injected into the rod inclusion (such as illustrated in FIG. 13B) and activated into gaseous MBs by the imaging transducer using the optimized vaporization parameters (2 cycle sinusoid at a center frequency of 5 MHZ, MI of 1.8; total activation duration of 2 seconds and the beam focused to z=13 mm). Immediately after vaporization, the imaging transducer acquired a US image of the MB-filled inclusion. Low frequency US at center frequencies of 850, 250 and 80 kHz were applied to the inclusion, with PNPs ranging 85-1000 kPa for the 850 kHz, 75-600 kPa for the 250 kHz and 25-200 kPa for the 80 kHz. The treatment duration was 1 second, at a PRF of 33 Hz and a pulse length of 0.5 ms. After the low frequency application, the imaging transducer acquired a US image of the inclusion. Post-processing of the captured images was used to calculate the change in contrast caused by MB destruction before and after the low frequency insonation process, using formula 2. Here, μᵢ was the mean value of the pixels within the region inside the NDs inclusion after the low frequency application process and μₒ was the mean value of the pixels in the same region before the process. The size of selected areas was adjusted based on the full width half max (FWHM) of each frequency that was used, to take the decrease in focal spot when increasing the frequency into account. The lateral and axial FWHM were 1.3 and 6.2 mm for the 850 kHz, 7 and 50 mm for the 250 kHz and 18.9 and 92.66 mm for the 80 kHz center frequencies, respectively.

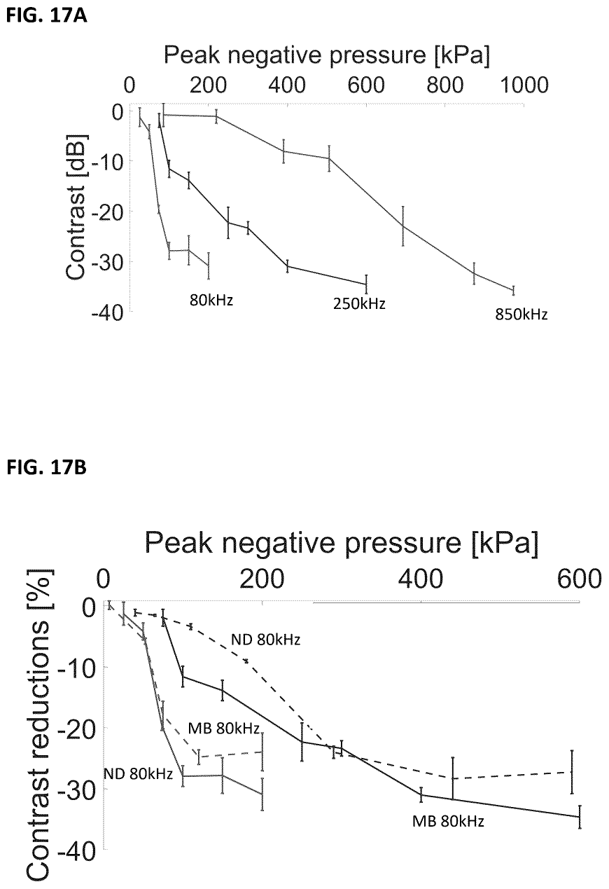

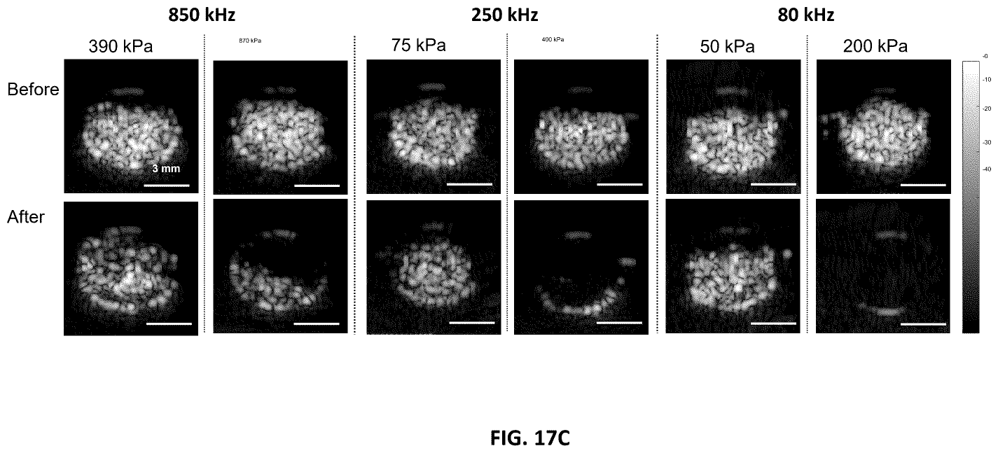

The purpose of the experiments was to confirm the numerical simulations by assessing the reduction in contrast as a function of the applied PNP upon low frequency insonation (FIG. 17A), and to compare standard MBs and vaporized NDs after insonation (FIG. 17B). After vaporization of the NDs into MBs, low frequency US at center frequencies of 850, 250 and 80 kHz were applied to the MB-filled inclusion. US images before and after the low frequency insonation were taken using the L7-4 imaging transducer. Once inertial cavitation occurred, the MB contrast diminishes. Therefore, the contrast was expected to decline when increasing the PNP. A contrast reduction to a value of ˜−30 dB was obtained at PNPs of 870, 400 and 200 kPa for 850, 250 and 80 kHz (FIG. 17A); examples of before and after images are presented in FIG. 17C. The contrast reduction slope was the steepest for the 80 kHz, consistent with the numerical simulations. Next, the behavior of standard MBs (as described in Example 2) was compared with vaporized NDs after application of low frequency US at frequencies of 250 and 80 kHz (FIG. 17B). The results indicated a similar contrast reduction between the MBs and the vaporized NDs (p>0.05).

### Example 15

Ex Vivo US and MB-Mediated Breast Tissue Ablation Assay using nanodroplets as MB source

A vial of MBs produced as described above in Example 2 was submerged in an isopropanol bath maintained between −8° C. and −10° C. Dry ice was used to cool the isopropanol. C₄ F₁₀ gas was injected into the vial using a syringe until it was hard to inject more gas into the vial and emulsion consistency indicating condensation was observed. NDs having an average diameter of 300 nanometers (nm) were produced with the above-noted method. To confirm the MB stability over this period, the size and concentration of the MBs was measured with a particle counter system (AccuSizer® FX-Nano, Particle Sizing Systems, Entegris, MA, USA) immediately after preparation.

In vitro US insonation setup (as shown in FIG. 3A or FIG. 13B), was used to conduct parametric experiments to determine appropriate US parameters for US-based activation of ND to form MBs. Based on the parametric experiments, an ND concentration of 0.66 μL/100μL, as well as US at a center frequency of 5 MHz and a PNP of 4085 kPa (MI=1.82), and having a pulse duration of 2 seconds, was used for the ex vivo experimentation using chicken breast sample.

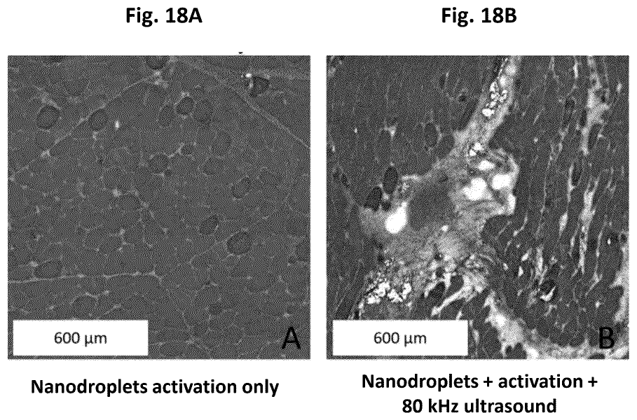

As shown in FIGS. 18A-18B, LE bubble histotripsy starting with administration and activation of NDs was tested in ex-vivo chicken breast samples, where the mechanical damage following the histotripsy was evaluated via histology. These experiments were performed as follows. An ND emulsion produced as described above was injected into a block of chicken breast, then activated through application of a US pulse of 2 seconds in duration having a center frequency of 5 MHz and a PNP of 4085 kPa. FIG. 18A shows an example cryosection of a chicken breast following ND injection and activation to form MBs, but without US-induced cavitation of the MBs. As can be seen in the image, there is not tissue damage observed. FIG. 6B shows an example cryosection of a chicken breast following ND injection and activation, and further followed by US-based cavitation of the MBs with a low-energy insonation with US having a center frequency of 80 KHz and a PNP of 250 kPa (MI=0.9). As can be seen in the figure, significant lesioning and tissue debulking was observed, indicating that ND-derived MBs had similar cavitation properties as non-ND-derived MBs used in the in vivo breast cancer ablation experiments shown in Example 4. As in the in vivo experiments, MBs cavitated using US with lower center frequencies were more effective in inducing tissue lesions. The following US parameters were tested to induce cavitation of ND-derived MBs in ex vivo chicken breasts: 250 kHz with 800kPa), 80 kHz with 250 kPa), and 850 kHz with 1250 kPa. Out of these parameters, the 80 kHz US (as shown in FIG. 18B) was most effective in inducing lesion formation, the 250 kHz US (not shown) had intermediate effect, and the 850 kHz US (not shown) was least effective in inducing lesion formation.

### Example 16

### Ex-Vivo Nonthermal Ablation

Fresh and unfrozen chicken livers were used in ex-vivo experiments. The livers were cut into 15 mm×7 mm pieces, and placed within the rod inclusion inside the agar mold (as illustrated in FIG. 13B). 200 μl of degassed water were injected into the inclusion, prior to placing the ex-vivo samples, to prevent air gaps along the US beam path. A 30 μL solution of 2×107 NDs and degassed PBS were injected into the center of each sample via an insulin micro syringe with a 31G needle under US imaging guidance to visualize the needle in the center of the sample prior to injection. The NDs solution was freshly prepared before each injection. The total treatment duration was 120 seconds, in which the imaging transducer activated the NDs, followed by the application of the low frequency treatment at a center frequency of 850, 250 or 80 kHz. Low frequency US was performed at an MI of 0.9, which corresponds to a PNP of 810, 440 and 250 kPa for the 850, 250 and 80 KHz frequencies, respectively. The low frequency treatments were performed at a PRF of 33 Hz and a pulse length of 0.5 ms. Several control groups were included: 1) no treatment control (NTC); Injection of diluted ND solution without additional treatment (“ND+only injection); injection of diluted ND solution, and application of the 2-cycle activation pulse using the imaging transducer for 120 seconds at a PRF of 20 Hz and an MI of 1.8 (“ND+Only activation”); injection of the diluted ND solution and application of low frequency therapeutic US at a frequency of 80 kHz, a PNP of 250 kPa and a total duration of 120 seconds (“ND+Only treatment”). After the US treatment, all samples were flash-frozen using liquid nitrogen and methyl butan and stored at −80° C. The frozen samples were cryo-sectioned to 30-μm-thick slices and stained with hematoxylin (Leica 3801542) and eosin (Leica 3801602) (H&E) according to the standard procedure. The H&E slides were scanned using the Aperio Versa 200 slide scanner (Leica Biosystems, Buffalo Grove, IL) at 20× optical magnification. Post-processing of the images was performed in ImageJ, to compare and quantify the damage in the form of lesions that were generated in the samples, for the different groups. Each image was cut into a square of the same size using the same scale and magnification. The lesion area of each image was outlined such that the pixels inside the marked area turned black and the rest of the pixels (outside the marked area) turned white (as shown in FIG. 15). Then, the lesion area in mm² was calculated as the number of black pixels multiplied by the pixel area. according to Equation 5:

Lesion area [mm\hat{ }2]=#black pixels*pixel area [mm\hat{ }2] (5)

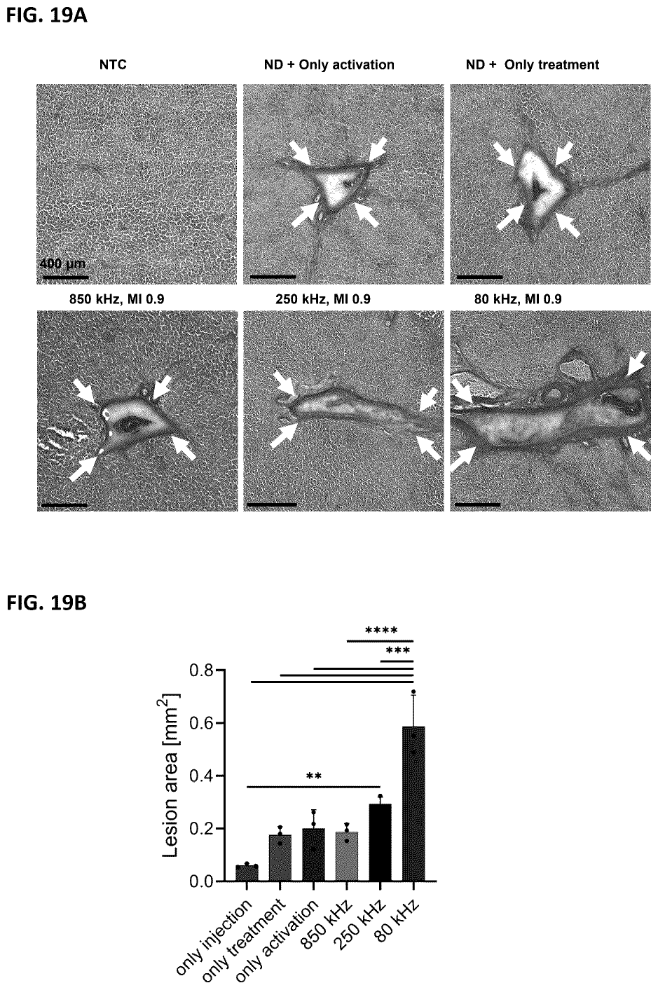

The two-step ND-mediated mechanical US surgery method was also evaluated in ex-vivo chicken liver samples. ND activation into MBs was performed with the optimized parameters, and subsequent MB detonation was performed at 850, 250 or 80 kHz, at an MI of 0.9. After the experiments, histology visualized the generated lesions (FIG. 19A). Significant lesions were observed for the 250 and 80 kHz treatments. Quantification of the lesion area for the different groups showed a 0.06±0.006 mm² lesion area for the ‘only injection’ group. Control groups of ‘only activation’ and ‘only treatment’ yielded lesion areas of 0.2±0.07 and 0.18±0.03 mm², respectively. The two-step method with the 850, 250 and 80 kHz frequencies yielded lesion areas of 0.19±0.03, 0.29±0.03, and 0.59±0.12 mm², respectively. Therefore, the generated lesion area was increased by a factor of 2 for the 80 kHz treatment compared to 250 kHz (p<0.001), and by a factor of 3.1 compared to the treatment with 850 kHz (p<0.0001). The lesion size of the treatment with a center frequency of 850 kHz was similar to the ‘ND+only treatment’ control and to the ‘ND+only activation’ control result.

### Example 17

### 3D Ultrasound Setup Experimental System

An US guided focused US was developed for the concurrent 3D activation and detonation of NDs. This hybrid experimental setup/system is shown in FIGS. 12A-B. The 3D US activation system includes a rotatory imaging US transducer (configured to emit high frequency US to convert th ND to MB), and a therapeutic transducer (configured to emit low energy US to implode the generated MBs). The 3D US system includes a rotatory imaging array that is controlled by a motorized rotary, and situated within a therapeutic transducer located at the bottom of a water tank. The imaging transducer (for example, IP104) is controlled by a programmable US system (Vantage 256, Verasonics Inc., Redmond, WA, USA). This transducer has 128 elements, with an element size of 7 mm×0.283 mm (height×width), a kerf width of 0.025 mm and operates at a center frequency of 3.47 MHz. 3D ND activation was done using this transducer by using the rotary motion while transmitting a 2-cycle pulse focused on the location of the injected NDs (distance of z=65 mm). The motorized rotary is an assembly that allows the user to rotate an imaging probe by +180° from its home position while examining a subject. The imaging probe rotary is controlled via MATLAB, allowing a precise control over the rotational position, speed and acceleration of the attached imaging probe.

The imaging transducer is also used for acquiring US images of the NDs before and after each optimization experiment.

The therapeutic transducer is a spherically focused single-element therapeutic transducer (H149, Sonic Concepts, Bothell, WA, USA) supporting multiple center frequencies including 75, 105, 200 and 600 kHz using custom matching networks. The therapeutic transducer focus was at a distance of 60 mm. The therapeutic transducer is operated using a transducer power output unit (TPO-200, Sonic Concepts, Bothell, WA, USA). Both imaging and therapeutic transducers PNPs were calibrated with a needle hydrophone (NH0200, Precision Acoustics, UK) in situ. In each experiment, an agarose phantom (prepared as detailed above) was placed at the focal spot of both the imaging and therapeutic transducers and contained either a diluted ND solution or the ex-vivo chicken liver samples inside the rod inclusion (as illustrated in FIG. 12C).

### Example 18

### Nanodroplet Vaporization Optimization Experiments in a 3D US System

The aim of these experiments was to optimize the vaporization process of NDs into MBs and compare between 2D activation and 3D activation. The imaging transducer (center frequency of 3.47 MHz) was used for both NDs activation and for the US images acquisition which were captured before after the activation. Parameters that were optimized are activation duration, peak negative pressure and concentration. A mixture of 0.666−2×10⁷ NDs/ml diluted with 300 μl degassed phosphate buffered saline (PBS) was injected into the rod inclusion in the agarose mold and filled the inclusion completely. Next, a 2-cycle excitation pulse (Sheeran et al. 2012b), with a pulse repetition frequency (PRF) of 20 Hz and a total duration of 2-6 seconds and PNPs ranging 1.2-3.8 MPa (MI of 0.65-2) was applied to the NDs inclusion to vaporize the NDs into MBs. Post-processing of the captured images was used to calculate the change in contrast before and after the vaporization process, using Formula 2 (Example 2).

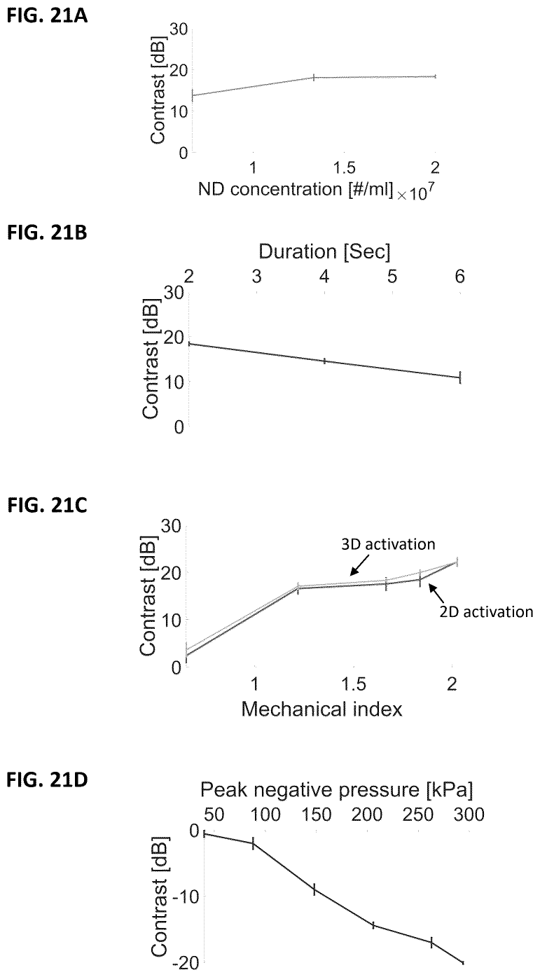

Optimization of the activation process was conducted using the 3D US system setup. NDs vaporization was performed with a rotating imaging transducer (IP104) at a center frequency of 3.47 MHz which allows 3D vaporization. The activation concentration (FIG. 21A), the activation duration (FIG. 21B) and the applied PNP (FIG. 21C) were optimized. US images were acquired before ND vaporization, where the inclusion appeared dark, and after ND vaporization, where the inclusion becomes hyperechoic as a result of the MB generation. The contrast difference was calculated and analyzed for each of the parameters. The contrast for ND concentration of 1.32×10⁷ NDs/mL and 2 × 107 NDs/mL was similar (FIG. 21A), therefore a concentration of 2×10⁷ NDs/mL was selected. The experiment was conducted without using the rotary motion. Next, 3D activation using different rotations speed which resulted in different activation durations was tested. An activation duration of 2 seconds yielded the highest contrast of 18.3 dB and was used for following experiments (FIG. 21B).

A comparison of 2D activation and 3D activation using different MI shows similar behavior. The contrast increased when increasing the activation pulse MI (FIG. 21C).

In order to maximize the contrast while operating below an MI of 1.9, an MI of 1.84, which yielded a contrast of 20 dB for a 3D activation and 18.4 dB for a 2D activation was consequently used.

### Example 19

### Low Frequency MB Insonation Optimization Experiments in 3D US System

In this experiment, a mixture of 2×10⁷ NDs/mL diluted in 300 μl of degassed PBS was injected into the rod inclusion and activated into gaseous MBs. Then, low frequency US (treatment duration of 2 s, PRF of 33 Hz and a pulse length of 0.5 ms at a center frequency of 105 kHz was applied on the vaporized NDs with PNPs ranging 40-300. US images were captured before and after low frequency application. The vaporization process was done using the optimized parameters (2 cycle sinusoid at a center frequency of 3.47 MHz, MI of 1.84; total activation duration of 2 seconds and the beam focused to z=65 mm). Post-processing of the captured images was used to calculate the change in contrast caused by MB destruction before and after the low frequency insonation process, using formula (2). Here, μᵢ was the mean value of the pixels within the region inside the NDs inclusion after the low frequency application process and μₒ was the mean value of the pixels in the same region before the process.

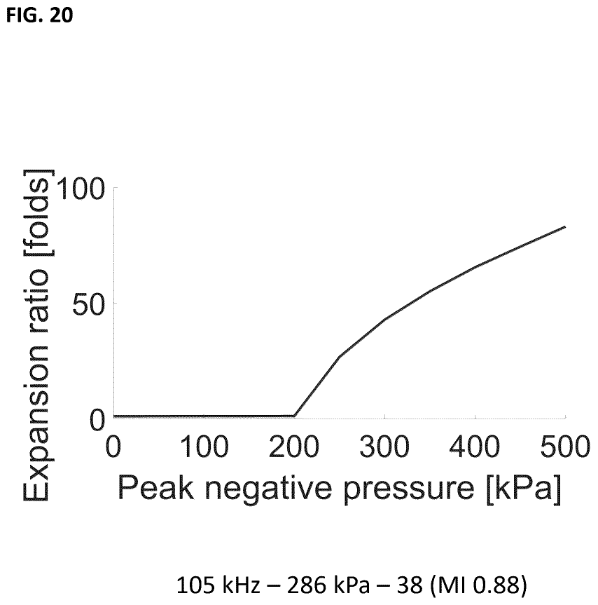

Theoretical prediction of microbubble expansion—Numerical stimulations were performed using the Marmottant model in order to estimate MB expansion ratio as a function of the PNP for a center frequency of 105 kHz (FIG. 20). The application of low frequency US is used in order to implode the vaporized NDs. MB transition to inertial cavitation occurs at expansion ratios above 3.5, therefore assessing the MB expansion ratio could indicate the range of pressures required for the process. When working with a MI of 0.9 (PNP of 290 kPa), the predicted expansion ratio was 38 (FIG. 20).

This experiment was performed in order to affirm numerical simulations by assessing the reduction in contrast as a function of the applied PNP upon low frequency insonation (FIG. 21D). Following activation of NDs into MBs, low frequency US excitation at a center frequency of 105 kHz was applied to the MB-filled inclusion. US images were taken before and after the treatment. When inertial cavitation occurs, the MB collapses, hence the contrast is expected to reduce as the PNP increases and more MBs collapse as can be seen in FIG. 21D.

### Example 20

### Ex-Vivo Nonthermal Ablation Using 3D US System

These experiments were conducted essentially as described above (Example 16). Briefly, Fresh and unfrozen chicken livers were cut into 15 mm×7 mm pieces and placed within the rod inclusion inside the agar mold. A 30 μL solution of 2×10⁷ NDs and degassed PBS were injected into the center of each sample via an insulin micro syringe with a 31G needle under US imaging guidance to visualize the needle in the center of the sample prior to injection. The treatment duration was 120 seconds in which the imaging transducer activated the NDs (using the optimized parameters), followed by application of the low frequency treatment (PRF of 33 Hz and a pulse length of 0.5 ms) at a center frequency of 105 kHz and PNP of 290 kPa (MI of 0.9). A comparison was made between 2D activation and 3D activation in addition to two control groups which included: 1) injection of diluted ND solution, and application of the 2-cycle 3D activation pulse using the rotatory imaging transducer for 120 seconds at a PRF of 20 Hz and an MI of 1.84 (“ND+Only activation”), 2) Injection of the diluted ND solution and application of low frequency therapeutic US at a frequency of 105 kHz, a PNP of 290 kPa and a total duration of 120 seconds (“ND+Only treatment”).

After the US treatment, all samples were flash-frozen using liquid nitrogen and methyl butane and stored at −80° C. The frozen samples were cryo-sectioned to 30-μm-thick slices and stained with hematoxylin (Leica 3801542) and eosin (Leica 3801602) (H&E) according to the standard procedure. The H&E slides were scanned using the Aperio Versa 200 slide scanner (Leica Biosystems, Buffalo Grove, IL) at 20× optical magnification. For comparison and quantification of the resulted lesions, post-processing of the scanned images was performed in ImageJ. Each image was cut into a square of the same size using the same scale and magnification. The lesion area of each image was outlined such that the pixels inside the marked area turned black and the rest of the pixels (outside the marked area) turned white. Then, the lesion area in mm² was calculated as the number of black pixels multiplied by the pixel area. according to formula 5.

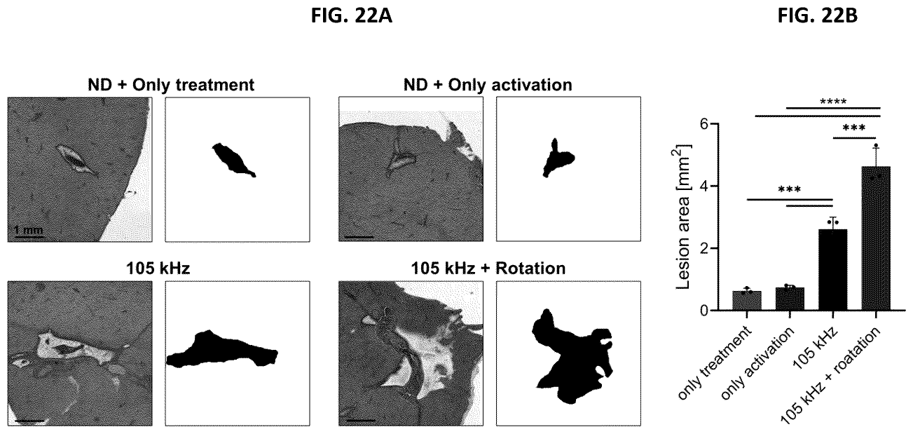

In order to evaluate the differences between 3D volumetric histotripsy and 2D histotripsy and to assess the morphology of each method, ex-vivo chicken liver samples were utilized. NDs activation into MBs was performed using the optimized parameters while simultaneously low frequency US at a center frequency of 105 kHz at a PNP of 290 kPa was applied on the vaporized NDs. The generated mechanical damage was then evaluated via histology. The results are presented in FIGS. 22A-22B. The results show that the treatment was successful for both methods. An average lesion area of 4.6±0.6 mm² was received for the 3D treatment, compared to 2.6±0.4 mm² when not using the rotatory approach (1.8 folds). Control groups of ‘only activation’ and ‘only treatment’ yielded lesion areas of 0.74±0.11 and 0.63±0.35 mm2, respectively.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1.-57. (canceled)

58. A method for inducing damage to a target tissue of a subject, the method comprising: administering microbubbles (MB) and/or nanobubbles (NBs) to the subject; and applying low frequency ultrasound (US) having a peak negative pressure (PNP) of about 400 kPa or less to the target tissue, to thereby induce damage to the target tissue.

59. The method according to claim 58, wherein the nanobubbles have an average diameter in the range of about 50-250 nm or in the range of about 110-230 nm.

60. The method according to claim 58, wherein the MBs are administered locally, into or in the vicinity of the target tissue.

61. The method according to claim 58, wherein the microbubbles and/or nanobubbles are essentially spherical.

62. The method according to claim 58, wherein the US is in the frequency of less than about 200 KHz.

63. The method according to claim 58, wherein the US is applied after a time interval from the administration of the MBs and/or NBs and wherein the time interval is at least 10 minutes.

64. A method for inducing damage to a target tissue of a subject, the method comprising:
administering nanodroplets to the subject;
applying high frequency ultrasound (US) to the target tissue, to thereby form microbubbles in the target tissue; and
applying low frequency US to the target tissue, to thereby induce tissue damage.

65. The method according to claim 64, wherein the high frequency US is applied using an ultrasound imaging transducer comprising a plurality of transducing elements and/or wherein the imaging transducer is situated within the therapeutic transducer.

66. The method according to claim 64, wherein the high frequency US is applied using a rotatory imaging US transducer, to thereby induce volumetric activation of the nanodroplets.

67. The method according claim 64, wherein the high-frequency US being characterized by a center frequency of about 1 MHz or more and a mechanical index of less than about 1.9.

68. The method according to claim 66, wherein the activation of the nanodroplets is facilitated in a 2-cycle excitation pulse.

69. The method according to claim 68, wherein the 2-cycle excitation pulse comprises a center frequency of about 1 MHz or more, and a peak negative pressure (PNP) of over about 2 MPa or about 3.4 MPa.

70. The method according to claim 64, wherein the low frequency US is characterized as having a peak negative pressure (PNP) of about 400 kPa or less and/or a frequency of below 1 MHz.

71. The method according to claim 64, wherein the low frequency US is applied after a time interval from the administration of the NDs and/or after a time interval after application of the high frequency US.

72. The method according to claim 64, wherein the tissue damage comprises: ablation, debulking and/or lesion of the tissue.

73. The method according to claim 64, wherein the target tissue is or comprises a tumor.

74. A system for inducing damage to a target tissue of a subject, the system comprising:
a high frequency imaging transducer configured to provide high frequency ultrasound characterized by a center frequency of 1 MHz or more and a mechanical index of less than about 1.9, towards the target tissue, said target tissue comprises nanodroplets (NDs); and
a low frequency focused ultrasound transmitter configured to emit low frequency ultrasound (US), having a peak negative pressure (PNP) of about 400 kPa or less, towards the target tissue,
wherein said high frequency ultrasound facilitates conversion of nanodroplets in the target tissue to microbubbles, and wherein the low frequency ultrasound causes said microbubbles to induce damage to the target tissue.

75. The system according to claim 74, wherein the imaging transducer comprises an array of transducing elements and/or wherein the imaging transducer is located within the therapeutic transducer.

76. The system according to claim 74, wherein the high frequency imaging transducer comprises a rotatory imaging transducer configured to provide 3D ultrasound energy.

77. The system according to claim 74, further comprising one or more of: a user interface, a controller, a power supply, a communication unit, or any combination thereof.
